SEQUENCE LISTING (52 sequences)

SEQ ID NO: 1 - protein, 1 aa, Artificial Sequence
  synthetic
M

SEQ ID NO: 2 - protein, 20 aa, Artificial Sequence
  synthetic
ALPVTALLLPLALLLHAARP

SEQ ID NO: 3 - protein, 116 aa, Artificial Sequence [sequence omitted]

SEQ ID NO: 4 - protein, 15 aa, Artificial Sequence
  synthetic
GGGGSGGGGSGGGGS

SEQ ID NO: 5 - protein, 112 aa, Artificial Sequence
  synthetic
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKR

SEQ ID NO: 6 - protein, 45 aa, Artificial Sequence
  synthetic
TTTPAPRPPTPAPTIASQPLSLRPEACRPAAGGAVHTRGLDFACD

SEQ ID NO: 7 - protein, 24 aa, Artificial Sequence
  synthetic
IYIWAPLAGTCGVLLLSLVITLYC

SEQ ID NO: 8 - protein, 42 aa, Artificial Sequence
  synthetic
KRGRKKLLYIFKQPFMRPVQTTQEEDGCSCRFPEEEEGGCEL

SEQ ID NO: 9 - protein, 112 aa, Artificial Sequence
  synthetic
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 42 aa, Artificial Sequence
  synthetic
AAAIEVMYPPPYLDNEKSNGTIIHVKGKHLCPSPLFPGPSKP

SEQ ID NO: 11 - protein, 68 aa, Artificial Sequence
  synthetic
FWVLVVVGGVLACYSLLVTVAFIIFWVRSKRSRLLHSDYMNMTPRRPGPTRKHYQPYAPPRDFAAYRS

SEQ ID NO: 12 - protein, 41 aa, Artificial Sequence
  synthetic
ALPVTALLLPLALLLHAARPMALPVTALLLPLALLLHAARP

SEQ ID NO: 13 - protein, 116 aa, Artificial Sequence
  synthetic
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 14 - protein, 111 aa, Artificial Sequence
  synthetic
DIQMTQSPSSLSASVGDRVTITCRASESVDNYGISFMNWFQQKPGKAPKLLIYAASNQGSGVPSRFSGSGSGTDFTLNISSLQPDDFATYYCQQSKEVPWTFGQGTKVEIK

SEQ ID NO: 15 - protein, 116 aa, Artificial Sequence; identical to SEQ ID NO: 13
  synthetic

SEQ ID NO: 16 - protein, 111 aa, Artificial Sequence
  synthetic
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 17 - protein, 5 aa, Artificial Sequence
  synthetic
GGGGS

SEQ ID NO: 18 - protein, 491 aa, Artificial Sequence
  synthetic
MALPVTALLLPLALLLHAARPEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 486 aa, Artificial Sequence
  synthetic
MALPVTALLLPLALLLHAARPEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 511 aa, Artificial Sequence
  synthetic
MALPVTALLLPLALLLHAARPMALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLNISSLQPDDFATYYCQQSKEVPWTFGQGTKVEIKTSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 510 aa, Artificial Sequence
  synthetic
MALPVTALLLPLALLLHAARPMALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLNISSLQPDDFATYYCQQSKEVPWTFGQGTKVEIKTSSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 488 aa, Artificial Sequence
  synthetic
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 487 aa, Artificial Sequence
  synthetic
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - DNA, 1476 bp, Artificial Sequence
  synthetic
atggccctgcctgtgacagccctgctgctgcccctggctctgctgctgcatgccgccagacctgaggtgcagctggtgcagtctggcgccgaagtgaagaaacccggcagcagcgtgaaggtgtcctgcaaggccagcggctacaccatcaccgacagcaacatccactgggtgcgccaggcccctggccagagcctggaatggatcggctacatctacccctacaacggcggcaccgactacaaccagaagttcaagaaccgggccaccctgaccgtggacaaccccaccaacaccgcctacatggaactgagcagcctgcggagcgaggacaccgccttctactactgcgtgaacggcaacccctggctggcctactggggccagggaaccctggtgacagtgtctagcggcggaggcggatctggagggggaggatctggcggcggaggaagcgacatccagctgacccagagccccagcaccctgagcgccagcgtgggcgacagagtgaccatcacctgtcgggccagcgagagcctggacaactacggcatccggtttctgacctggttccagcagaagcccggcaaggcccccaagctgctgatgtacgccgccagcaatcagggcagcggcgtgcccagcagattcagcggctctggcagcggaaccgagttcaccctgaccatcagcagcctgcagcccgacgacttcgccacctactactgccagcagaccaaagaggtgccctggtccttcggccagggcaccaaggtggaagtgaagcggactagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 25 - DNA, 1479 bp, Artificial Sequence
  synthetic
atggccctgcctgtgacagccctgctgctgcccctggctctgctgctgcatgccgccagacctgaggtgcagctggtgcagtctggcgccgaagtgaagaaacccggcagcagcgtgaaggtgtcctgcaaggccagcggctacaccatcaccgacagcaacatccactgggtgcgccaggcccctggccagagcctggaatggatcggctacatctacccctacaacggcggcaccgactacaaccagaagttcaagaaccgggccaccctgaccgtggacaaccccaccaacaccgcctacatggaactgagcagcctgcggagcgaggacaccgccttctactactgcgtgaacggcaacccctggctggcctactggggccagggaaccctggtgacagtgtctagcggcggaggcggatctggagggggaggatctggcggcggaggaagcgacatccagctgacccagagccccagcaccctgagcgccagcgtgggcgacagagtgaccatcacctgtcgggccagcgagagcctggacaactacggcatccggtttctgacctggttccagcagaagcccggcaaggcccccaagctgctgatgtacgccgccagcaatcagggcagcggcgtgcccagcagattcagcggctctggcagcggaaccgagttcaccctgaccatcagcagcctgcagcccgacgacttcgccacctactactgccagcagaccaaagaggtgccctggtccttcggccagggcaccaaggtggaagtgaagcggactagttccggagccgccgccatcgaagtgatgtacccccctccctacctggataacgagaagagcaacggcaccatcatccacgtgaagggaaagcacctgtgtcccagccccctgtttcccggccctagcaagcccttctgggtgctggtggtggtcggcggagtgctggcctgctacagcctcctggtgaccgtggccttcatcatcttctgggtgaggagcaagaggtccaggctgctgcacagcgactacatgaatatgacccccagaaggcccggccccaccagaaagcactatcagccctacgccccccccagggactttgccgcctacaggagcagggtgaagttcagcagatccgccgatgcccctgcttaccagcagggccagaaccagctgtataacgagctgaacctgggcaggagggaggaatacgacgtgctggataagaggaggggaagggaccccgagatgggcggaaagcccaggaggaagaacccccaggagggcctgtacaatgagctgcagaaagacaagatggccgaggcctacagcgagatcggcatgaagggcgagaggaggaggggcaagggccatgacggcctgtaccaaggcctgtccaccgccaccaaggatacctacgacgccctgcacatgcaggccctgcctcccaggggatcctaa

SEQ ID NO: 26 - DNA, 1536 bp, Artificial Sequence
  synthetic
atggccctgcctgtgacagccctgctgctgcccctggctctgctgctgcatgccgccagacctatggctctgcccgtgaccgctctcctcctgccactggcactgctcctccacgctgctagaccccaggtgcagctggtgcagtctggcgccgaagtgaagaaacccggcagcagcgtgaaggtgtcctgcaaggccagcggctacaccttcaccgactacaacatgcactgggtgcgccaggctccaggccagggactggaatggatcggctacatctacccctacaacggcggcaccggctacaaccagaagttcaagagcaaggccaccatcaccgccgacgagagcaccaacaccgcctacatggaactgagcagcctgcggagcgaggacaccgccgtgtactactgcgccagaggcagacccgccatggactactggggccagggcaccctggtgacagtgtctagcggaggcggaggctctggcggcggaggaagtggcggaggcggcagcgatatccagatgacccagagccccagcagcctgagcgccagcgtgggcgacagagtgaccatcacctgtcgggccagcgagagcgtggacaactacggcatcagcttcatgaactggttccagcagaagcccggcaaggcccccaagctgctgatctacgccgccagcaatcagggcagcggcgtgcccagcagattcagcggctctggcagcggcaccgacttcaccctgaacatcagcagcctgcagcccgacgacttcgccacctactactgccagcagagcaaagaggtgccctggaccttcggacagggcaccaaggtggaaatcaagactagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 27 - DNA, 1539 bp, Artificial Sequence
  synthetic
atggccctgcctgtgacagccctgctgctgcccctggctctgctgctgcatgccgccagacctatggctctgcccgtgaccgctctcctcctgccactggcactgctcctccacgctgctagaccccaggtgcagctggtgcagtctggcgccgaagtgaagaaacccggcagcagcgtgaaggtgtcctgcaaggccagcggctacaccttcaccgactacaacatgcactgggtgcgccaggctccaggccagggactggaatggatcggctacatctacccctacaacggcggcaccggctacaaccagaagttcaagagcaaggccaccatcaccgccgacgagagcaccaacaccgcctacatggaactgagcagcctgcggagcgaggacaccgccgtgtactactgcgccagaggcagacccgccatggactactggggccagggcaccctggtgacagtgtctagcggaggcggaggctctggcggcggaggaagtggcggaggcggcagcgatatccagatgacccagagccccagcagcctgagcgccagcgtgggcgacagagtgaccatcacctgtcgggccagcgagagcgtggacaactacggcatcagcttcatgaactggttccagcagaagcccggcaaggcccccaagctgctgatctacgccgccagcaatcagggcagcggcgtgcccagcagattcagcggctctggcagcggcaccgacttcaccctgaacatcagcagcctgcagcccgacgacttcgccacctactactgccagcagagcaaagaggtgccctggaccttcggacagggcaccaaggtggaaatcaagactagttccggagccgccgccatcgaagtgatgtacccccctccctacctggataacgagaagagcaacggcaccatcatccacgtgaagggaaagcacctgtgtcccagccccctgtttcccggccctagcaagcccttctgggtgctggtggtggtcggcggagtgctggcctgctacagcctcctggtgaccgtggccttcatcatcttctgggtgaggagcaagaggtccaggctgctgcacagcgactacatgaatatgacccccagaaggcccggccccaccagaaagcactatcagccctacgccccccccagggactttgccgcctacaggagcagggtgaagttcagcagatccgccgatgcccctgcttaccagcagggccagaaccagctgtataacgagctgaacctgggcaggagggaggaatacgacgtgctggataagaggaggggaagggaccccgagatgggcggaaagcccaggaggaagaacccccaggagggcctgtacaatgagctgcagaaagacaagatggccgaggcctacagcgagatcggcatgaagggcgagaggaggaggggcaagggccatgacggcctgtaccaaggcctgtccaccgccaccaaggatacctacgacgccctgcacatgcaggccctgcctcccaggggatcctaa

SEQ ID NO: 28 - DNA, 1467 bp, Artificial Sequence
  synthetic
atggctctgcccgtcacagctctgctgctgcctctggccctgctgctgcacgccgccagacctcaggtgcagctcgtgcagagcggcgctgaggtgaagaaacctggcagcagcgtgaaggtgagctgcaaggcctccggctacaccttcaccgactacaacatgcactgggtgaggcaagcccctggccagggactggagtggatcggctacatctacccttacaacggcggcacaggctacaaccagaagttcaagtccaaggccaccatcaccgccgatgagtccaccaataccgcctacatggagctcagcagcctgaggtccgaggacacagccgtctactactgcgccaggggcaggcccgctatggactactggggccagggcaccctggtgacagtgagctctggtggcggcggatccggcggcggcggcagcggcggcggcggctccgacattcagatgacccagagccctagcagcctgagcgcttccgtgggagacagggtgaccatcacatgcagggcctccgagagcgtggacaattacggcatcagcttcatgaactggttccagcagaagcccggcaaggcccccaaactgctgatctatgccgccagcaatcagggctccggcgtgcctagcaggttttccggcagcggcagcggcaccgactttaccctgaccatctccagcctgcagcctgacgatttcgccacctactactgccagcagagcaaggaggtgccttggacctttggacagggcacaaaggtggagatcaagtccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 29 - DNA, 1470 bp, Artificial Sequence
  synthetic
atggctctgcccgtcacagctctgctgctgcctctggccctgctgctgcacgccgccagacctcaggtgcagctcgtgcagagcggcgctgaggtgaagaaacctggcagcagcgtgaaggtgagctgcaaggcctccggctacaccttcaccgactacaacatgcactgggtgaggcaagcccctggccagggactggagtggatcggctacatctacccttacaacggcggcacaggctacaaccagaagttcaagtccaaggccaccatcaccgccgatgagtccaccaataccgcctacatggagctcagcagcctgaggtccgaggacacagccgtctactactgcgccaggggcaggcccgctatggactactggggccagggcaccctggtgacagtgagctctggtggcggcggatccggcggcggcggcagcggcggcggcggctccgacattcagatgacccagagccctagcagcctgagcgcttccgtgggagacagggtgaccatcacatgcagggcctccgagagcgtggacaattacggcatcagcttcatgaactggttccagcagaagcccggcaaggcccccaaactgctgatctatgccgccagcaatcagggctccggcgtgcctagcaggttttccggcagcggcagcggcaccgactttaccctgaccatctccagcctgcagcctgacgatttcgccacctactactgccagcagagcaaggaggtgccttggacctttggacagggcacaaaggtggagatcaagtccggagccgccgccatcgaagtgatgtacccccctccctacctggataacgagaagagcaacggcaccatcatccacgtgaagggaaagcacctgtgtcccagccccctgtttcccggccctagcaagcccttctgggtgctggtggtggtcggcggagtgctggcctgctacagcctcctggtgaccgtggccttcatcatcttctgggtgaggagcaagaggtccaggctgctgcacagcgactacatgaatatgacccccagaaggcccggccccaccagaaagcactatcagccctacgccccccccagggactttgccgcctacaggagcagggtgaagttcagcagatccgccgatgcccctgcttaccagcagggccagaaccagctgtataacgagctgaacctgggcaggagggaggaatacgacgtgctggataagaggaggggaagggaccccgagatgggcggaaagcccaggaggaagaacccccaggagggcctgtacaatgagctgcagaaagacaagatggccgaggcctacagcgagatcggcatgaagggcgagaggaggaggggcaagggccatgacggcctgtaccaaggcctgtccaccgccaccaaggatacctacgacgccctgcacatgcaggccctgcctcccaggggatcctaa

SEQ ID NO: 30 - DNA, 7691 bp, Artificial Sequence
  synthetic
gacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattgggggggtacagtgcaggggaaagaatagtagacataatagcaacagacatacaaactaaagaattacaaaaacaaattacaaaaattcaaaattttcgggtttattacagggacagcagagatccagtttggctgcatacgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgag

SEQ ID NO: 31 - protein, 34 aa, Artificial Sequence
  synthetic
SGVPSRFSGSGSGTDFTLTISSLQPDDFATYYCQ

SEQ ID NO: 32 - protein, 34 aa, Artificial Sequence
  synthetic
SGVPSRFSGSGSGTDFTLNISSLQPDDFATYYCQ

SEQ ID NO: 33 - protein, 26 aa, Artificial Sequence
  synthetic
AYMELSSLRSEDTAFYYCVNGNPWLA

SEQ ID NO: 34 - protein, 26 aa, Artificial Sequence
  synthetic
AYMELSSLRSEDTDFYYCVNGNPWLA

SEQ ID NO: 35 - protein, 246 aa, Artificial Sequence
  synthetic
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKTSSG

SEQ ID NO: 36 - protein, 243 aa, Artificial Sequence
  synthetic
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKR

SEQ ID NO: 37 - protein, 14 aa, Artificial Sequence
  synthetic
RVKFSRSADAPAYQ

SEQ ID NO: 38 - protein, 4 aa, Artificial Sequence
  synthetic
TSSG

SEQ ID NO: 39 - protein, 2 aa, Artificial Sequence
  synthetic
SG

SEQ ID NO: 40 - DNA, 708 bp, Artificial Sequence
  synthetic
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 41 - protein, 5 aa, Artificial Sequence
  synthetic
DYNMH

SEQ ID NO: 42 - protein, 19 aa, Artificial Sequence
  synthetic
YIYPYNGGTGYNQKFKSKA

SEQ ID NO: 43 - protein, 10 aa, Artificial Sequence
  synthetic
GRPAMDYWGQ

SEQ ID NO: 44 - protein, 15 aa, Artificial Sequence
  synthetic
RASESVDNYGISFMN

SEQ ID NO: 45 - protein, 7 aa, Artificial Sequence
  synthetic
AASNQGS

SEQ ID NO: 46 - protein, 9 aa, Artificial Sequence
  synthetic
QQSKEVPWT

SEQ ID NO: 47 - protein, 8 aa, Artificial Sequence
  synthetic
GYTITDSN

SEQ ID NO: 48 - protein, 8 aa, Artificial Sequence
  synthetic
IYPYNGGT

SEQ ID NO: 49 - protein, 9 aa, Artificial Sequence
  synthetic
VNGNPWLAY

SEQ ID NO: 50 - protein, 10 aa, Artificial Sequence
  synthetic
ESLDNYGIRF

SEQ ID NO: 51 - protein, 3 aa, Artificial Sequence
  synthetic
AAS

SEQ ID NO: 52 - protein, 9 aa, Artificial Sequence
  synthetic
QQTKEVPWS

(19) United States
(12) Patent Application Publication
(10) Pub. No.: US 2021/0017277 A1
(43) Pub. Date: Jan. 21, 2021

(54) ANTI-CD33 CHIMERIC ANTIGEN RECEPTORS AND THEIR USES

(71) Applicant: The United States of America,as represented by the Secretary,Department of Health and Human Services, Bethesda, MD (US)

(72) Inventors: Haiying Qin, Potomac, MD (US); Terry J. Fry, Bethesda, MD (US)

(73) Assignee: The United States of America,as represented by the Secretary,Department of Health and Human Services, Bethesda, MD (US)

(21) Appl. No.: 16/980,205

(22) PCT Filed: Mar. 14, 2019
(86) PCT No.: PCT/US2019/022309

§ 371 Date: Sep. 11, 2020

Related U.S. Application Data
(60) Provisional application No. 62/643,015, filed on Mar. 14, 2018.

(51) Int. Cl. C07K 16/28; C07K 14/705; C07K 14/725; G01N 33/574; A61P 35/02

(52) U.S. Cl. CPC C07K 16/2803; C07K 14/70578; C07K 14/70521; C07K 14/7051; C07K 2319/02; G01N 33/57426; A61P 35/02; C07K 2317/622; C07K 2319/03; G01N 33/57492

(57) ABSTRACT

Embodiments of the invention provide chimeric antigen receptors (CARs) having antigenic specificity for CD33. Nucleic acids, recombinant expression vectors, host cells, populations of cells, and pharmaceutical compositions relating to the CARs are disclosed. Methods of detecting the presence of cancer in a mammal and methods of treating or preventing cancer in a mammal are also disclosed.

### CROSS-REFERENCE TO RELATED APPLICATION

This patent application claims the benefit of U.S. Provisional Patent Application No. 62/643,015, filed Mar. 14, 2018, which is incorporated by reference herein in its entirety.

### STATEMENT REGARDING FEDERALLY SPONSORED RESEARCH AND DEVELOPMENT

This invention was made with Government support under project number ZIA BC 011565 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: one 69,707 Byte ASCII (Text) file named “741580_ST25.txt,” dated Mar. 14, 2019.

### BACKGROUND OF THE INVENTION

Acute myelogenous leukemia is a highly aggressive acute leukemia, representing the second most common leukemia occurring in children and adolescents and young adults (AYAs). Despite current treatment regimens, which include intensive cycles of multi-agent chemotherapy, and frequently consolidation with allogeneic donor stem cell transplantation to achieve cure, only 60% of children and AYAs with AML will be achieve long-term remission. New therapeutic strategies are needed to increase remission rates, decrease relapse and to improve overall survival.

### BRIEF SUMMARY OF THE INVENTION

Embodiments of the invention provide chimeric antigen receptors (CARs) comprising an antigen binding domain specific for CD33, a transmembrane domain, and an intracellular T cell signaling domain. Another embodiment of the invention provides CAR constructs comprising the amino acid sequences as described herein.

Further embodiments of the invention provide related nucleic acids, recombinant expression vectors, host cells, populations of cells, and pharmaceutical compositions relating to the CAR constructs of the invention.

Additional embodiments of the invention provide methods of detecting the presence of cancer in a mammal and methods of treating or preventing cancer in a mammal.

### BRIEF DESCRIPTION OF THE DRAWINGS

FIGS. 1A and 1B present diagrams of certain CAR embodiments of the invention. Mylo: Mylotarg, humanized antibody hP67.6 targeting human CD33. M195: humanized monoclonal murine IgG2a antibody (M195) targeting human CD33 from a mouse immunized with live human leukemic myeloblasts. Hu195: humanized antibody targeting human CD33.

FIGS. 2A-2F present graphs showing transduction efficiency of CARs.

FIGS. 3A and 3B present graphs showing flow cytometric analysis of CD33 and CD123 target antigen expression (indicated as fluorescence intensity) on Leukemia cells.

FIGS. 4A-4F present graphs showing cytokine production by CD33 and CD123 CAR T cells following in vitro stimulation. CD33 or CD123 CAR transduced T cells were incubated with target leukemia cells as indicated in the figure. The interferon gamma or IL-2 level in the supernatant were detected by ELISA.

FIGS. 5A-5C present graphs of an IncuCyte killing assay. CD33 CAR transduced T cells were incubated with target leukemia cells as indicated in the figure. The differences of the live leukemia cells relative to the original plated cells were plotted.

FIGS. 5D and 5E present graphs of an IncuCyte killing assay. CD123 CAR transduced T cells were incubated with target leukemia cells as indicated in the figure. The differences of the live leukemia cell were normalized to the tumor only control. 5D: Killing of MOLM14 cell. 5E: Killing of THP1 cells.

FIGS. 6A-6E present bioluminescent images that were used to track the leukemia progression with different treatments in vivo, as shown. The anti-CD19 CAR is non-specific to the CD33 antigen.

FIG. 7A shows bioluminescent images used to track the leukemia progression with different treatments in vivo. 1 million of PDX Leukemia cell JMM117 were injected on day −7 into the NSG mice. The mice were treated with CAR T cells on day 7.

FIGS. 7B and 7C are graphs that show (FIG. 7B) human AML JMML117 cells and (FIG. 7C) CD33 CAR T cells in the spleen at week two. The numbers for both figures are as presented in the legend of FIG. 7B.

FIG. 8 is a graph showing flow cytometric analysis of CD33 target antigen expression (indicated as fluorescence intensity) on Leukemia cells. The abbreviations are as described in Example 2.

FIGS. 9A and 9B are bar graphs showing cytokines production by CD33Hu195-CD28Z CAR T cells following in vitro stimulation. CD33Hu195-CD28Z CAR-transduced T cells were incubated with target leukemia cells as indicated in the figure for 16 hours. The interferon gamma or IL-2 level in the supernatant were detected by ELISA.

FIG. 10 presents bioluminescent images that were used to track the leukemia progression with different treatments in vivo, as shown. One million leukemia cells of MOLM14 were injected on day −7 into NSG mice. The mice were treated with saline, untreated, or treated with CAR T cells 7 days later (number of cells listed above the columns of images). Darker regions represent greater tumor burden. “Scale” relates to the florescence intensity, which is based on the display range of the value (if the range is placed at the low value, the florescence intensity will look very high, but if the range is placed at the high value, the intensity will looks dim).

FIGS. 11A and 11B: validation of the CD33Hu195-CD28z clinical vector. FIG. 11A: CD33 CAR Expression Detection with Biotinylated Human Siglec-3. FIG. 11B: Bioluminescent image used to track the leukemia progression with different treatments in vivo. One million of Leukemia cell MOLM14 were injected on day 0 into the NSG mice. The mice were treated with 5E6 CAR T cells on day 3.

FIGS. 12A-12C: Effects of CAR co-stimulation Domain on Cellular Metabolism. CD33.2-28z and CD33.2-BBz CAR T cells were co-incubation with MOML14 and 7 days later tested on the metabolic features with Seahorse machine. FIG. 12A (top curve is 5 day-CD33.2-28, bottom curve is 5 day-CD33.2-BB): The oxygen consumption rates (OCRs) of CD33.2-28z and CC33.2-BBz CAR T cells on day 7 under basal metabolic conditions and in response to mitochondrial inhibitors. FIG. 12B (left is CD33.2-28, right is CD33.2-BB): Basal OCR levels vs maximum respiratory levels. FIG. 12C (left is CD33.2-28, right is CD33.2-BB): OCR for proton leak-linked and ATP production-linked.

FIGS. 12D-124F: Effects of CAR co-stimulation Domain on Cell Energy Phenotype. CD33.2-28z and CD33.2-BBz CAR T cells were co-incubation with MOML14 and 7 days later tested on the cell energy phenotype with Seahorse machine (FIG. 12D, left curve is CD33.2-28, right curve is CD33.2-BB) Cell Energy Phenotype. (FIG. 12E, left is CD33.2-28, right is CD33.2-BB) The Oxygen consumption rate. (FIG. 12F, left is CD33.2-28, right is CD33.2-BB) Extracellular aridification rate.

FIG. 13 presents bioluminescent images that were used to track the leukemia progression with different treatments in vivo, as shown. Darker regions represent greater tumor burden.

### DETAILED DESCRIPTION OF THE INVENTION

Acute Myeloid Leukemia (AML) is an aggressive malignancy that is normally treated using intensive cytotoxic chemotherapeutic regimens with limited alternative therapeutic options when the disease becomes refractory to cytotoxic chemotherapy.

A CAR is an artificially constructed hybrid protein or polypeptide containing the antigen binding domain of one or more antibodies (e.g., single chain variable fragment (scFv)) linked to T-cell signaling domains. Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains. The phrases “antigen(ic) specificity” and “elicit antigen-specific response,” as used herein, means that the CAR can specifically bind to and immunologically recognize antigen, such that binding of the CAR to the antigen elicits an immune response. CD33 is expressed on the surface of the vast majority of AML blasts and chronic myeloid leukemia in blast crisis. It is also aberrantly expressed on a subset of T cell acute lymphoblastic leukemias. Normal tissue expression is restricted to normal myeloid cells.

An embodiment of the invention provides a CAR comprising an anti-CD33 antigen binding domain of hP67.6 (Cowan et al., Front. Biosci. (Landmark Ed.), 18: 1311-1334 (2013) and U.S. Pat. No. 5,739,116, each incorporated by reference herein), M195 (Co et al., J. Immunol., 148: 1149-1154 (1992), incorporated by reference herein), or Hu195 (Co et al., supra). The antigen binding domain specifically binds to CD33. In this regard, a preferred embodiment of the invention provides CARs comprising an anti-CD33 antigen-binding domain comprising, consisting of, or consisting essentially of, a single chain variable fragment (scFv) of the antigen binding domain of hP67.6, M195, or Hu195.

The anti-CD33 antigen binding domain may comprise a light chain variable region and/or a heavy chain variable region, e.g. of hP67.6. In an embodiment of the invention, the heavy chain variable region comprises a CDR1 region, a CDR2 region, and a CDR3 region. In an embodiment of the invention, the light chain variable region of the anti-CD33 antigen binding domain may comprise a light chain CDR1 region, a light chain CDR2 region, and a light chain CDR3.

The heavy chain variable region of the anti-CD33 antigen binding domain may comprise, consist of, or consist essentially of the amino acid sequence of SEQ ID NO: 3. The light chain variable region of the anti-CD33 antigen binding domain may comprise, consist of, or consist essentially of the amino acid sequence of SEQ ID NO: 5. Accordingly, in an embodiment of the invention, the anti-CD33 antigen binding domain comprises a heavy chain variable region comprising the amino acid sequence of SEQ ID NO: 3 and/or a light chain variable region comprising the amino acid sequence of SEQ ID NO: 5. Preferably, the anti-CD33 antigen binding domain comprises the amino acid sequence of SEQ ID NOs: 3 and 5.

The anti-CD33 antigen binding domain may comprise a light chain variable region and/or a heavy chain variable region, e.g. of M195. In an embodiment of the invention, the heavy chain variable region comprises a CDR1 region, a CDR2 region, and a CDR3 region. In an embodiment of the invention, the light chain variable region of the anti-CD33 antigen binding domain may comprise a light chain CDR1 region, a light chain CDR2 region, and a light chain CDR3.

The heavy chain variable region of the anti-CD33 antigen binding domain may comprise, consist of, or consist essentially of the amino acid sequence of SEQ ID NO: 13. The light chain variable region of the anti-CD33 antigen binding domain may comprise, consist of, or consist essentially of the amino acid sequence of SEQ ID NO: 14. Accordingly, in an embodiment of the invention, the anti-CD33 antigen binding domain comprises a heavy chain variable region comprising the amino acid sequence of SEQ ID NO: 13 and/or a light chain variable region comprising the amino acid sequence of SEQ ID NO: 14. Preferably, the anti-CD33 antigen binding domain comprises the amino acid sequence of SEQ ID NOs: 13 and 14.

The anti-CD33 antigen binding domain may comprise a light chain variable region and/or a heavy chain variable region, e.g. of Hu195. In an embodiment of the invention, the heavy chain variable region comprises a CDR1 region, a CDR2 region, and a CDR3 region. In an embodiment of the invention, the light chain variable region of the anti-CD33 antigen binding domain may comprise a light chain CDR1 region, a light chain CDR2 region, and a light chain CDR3.

The heavy chain variable region of the anti-CD33 antigen binding domain may comprise, consist of, or consist essentially of the amino acid sequence of SEQ ID NO: 15. The light chain variable region of the anti-CD33 antigen binding domain may comprise, consist of, or consist essentially of the amino acid sequence of SEQ ID NO: 16. Accordingly, in an embodiment of the invention, the anti-CD33 antigen binding domain comprises a heavy chain variable region comprising the amino acid sequence of SEQ ID NO: 15 and/or a light chain variable region comprising the amino acid sequence of SEQ ID NO: 16. Preferably, the anti-CD33 antigen binding domain comprises the amino acid sequence of SEQ ID NOs: 15 and 16.

Within Hu195, the sequence SGVPSRFSGSGSGTDFTLTISSLQPDDFATYYCQ (SEQ ID NO: 31) may instead be SGVPSRFSGSGSGTDFTLNISSLQPDDFATYYCQ (SEQ ID NO: 32). Within Mylo, the sequence AYMELSSLRSEDTAFYYCVNGNPWLA (SEQ ID NO: 33) may instead be AYMELSSLRSEDTDFYYCVNGNPWLA (SEQ ID NO: 34).

The anti-CD33 antigen binding domain may comprise any antigen binding portion of the anti-CD33 antibody. The antigen binding portion can be any portion that has at least one antigen binding site, such as Fab, F(ab′)₂, dsFv, scFv, diabodies, and triabodies. Preferably, the antigen binding portion is a single-chain variable region fragment (scFv) antibody fragment. An scFv is a truncated Fab fragment including the variable (V) domain of an antibody heavy chain linked to a V domain of a light antibody chain via a synthetic peptide linker, which can be generated using routine recombinant DNA technology techniques. Similarly, disulfide-stabilized variable region fragments (dsFv) can be prepared by recombinant DNA technology.

In an embodiment of the invention, the light chain variable region and the heavy chain variable region of the anti-CD33 antigen binding domain can be joined to each other by a linker. The linker may comprise any suitable amino acid sequence. In an embodiment of the invention, the linker is a Gly/Ser linker from about 1 to about 100, from about 3 to about 20, from about 5 to about 30, from about 5 to about 18, or from about 3 to about 8 amino acids in length and consists of glycine and/or serine residues in sequence. Accordingly, the Gly/Ser linker may consist of glycine and/or serine residues. Preferably, the Gly/Ser linker comprises the amino acid sequence of GGGGS (SEQ ID NO: 17), and multiple SEQ ID NOs: 17 may be present within the linker. Any linker sequence may be used as a spacer between the antigen binding domain and the transmembrane domain.

In an embodiment, the anti-CD33 antigen binding domain comprises a light chain variable region, a heavy chain variable region, and a linker. In this regard, an embodiment of the anti-CD33 antigen binding domain comprising a light chain variable region, a heavy chain variable region, and the linker comprises, consists of, or consists essentially of, all of SEQ ID NOs: 3, 4, and 5; 13, 4, and 14; or 15, 4, and 16.

In an embodiment, the antigen binding domain comprises one or more leader sequences (signal peptides). In an embodiment of the invention, the leader sequence may be positioned at the amino terminus of the anti-CD33 CAR within the CAR construct. The leader sequence may comprise any suitable leader sequence, e.g., any CAR described herein may comprise any leader sequence as described herein. In an embodiment, the leader sequence comprises, consists of, or consists essentially of the amino acid sequence of SEQ ID NO: 2 or SEQ ID NO: 12. In an embodiment of the invention, while the leader sequence may facilitate expression of the released CARs on the surface of the cell, the presence of the leader sequence in an expressed CAR is not necessary in order for the CAR to function. In an embodiment of the invention, upon expression of the CAR on the cell surface, the leader sequence may be cleaved off. Accordingly, in an embodiment of the invention, the released CARs lack a leader sequence. In an embodiment of the invention, the CARs within the CAR construct lack a leader sequence.

In an embodiment of the invention, the CAR construct comprises a hinge domain. In an embodiment of the invention, the hinge domain is a CD8 hinge domain. In a preferred embodiment, the CD8 hinge domain is human. Preferably, the CD8 hinge domain comprises, consists of, or consists essentially of SEQ ID NO: 6. In an embodiment of the invention, the hinge domain is a CD28 hinge domain. In a preferred embodiment, the CD28 hinge domain is human. Preferably, the CD28 hinge domain comprises, consists of, or consists essentially of SEQ ID NO: 10.

In an embodiment of the invention, the CAR construct comprises a transmembrane (TM) domain. In an embodiment of the invention, the TM domain is a CD8 TM domain. In a preferred embodiment, the CD8 TM domain is human. Preferably, the CD8 TM domain comprises, consists of, or consists essentially of SEQ ID NO: 7. In an embodiment of the invention, the TM domain is a CD28 TM domain. In a preferred embodiment, the CD28 TM domain is human. Preferably, the CD28 TM domain comprises, consists of, or consists essentially of SEQ ID NO: 11.

In an embodiment of the invention, the CAR construct comprises an intracellular T cell signaling domain. In an embodiment of the invention, the intracellular T cell signaling domain comprises a 4-1BB intracellular T cell signaling sequence. 4-1BB, also known as CD137, transmits a potent costimulatory signal to T cells, promoting differentiation and enhancing long-term survival of T lymphocytes. Preferably, the 4-1BB intracellular T cell signaling sequence is human. In a preferred embodiment, the 4-1BB intracellular T cell signaling sequence comprises, consists of, or consists essentially of the amino acid sequence of SEQ ID NO: 8.

In an embodiment of the invention, the intracellular T cell signaling domain comprises a CD3 zeta (ζ) intracellular T cell signaling sequence. CD3ζ associates with TCRs to produce a signal and contains immunoreceptor tyrosine-based activation motifs (ITAMs). Preferably, the CD3ζ intracellular T cell signaling sequence is human. In a preferred embodiment, the CD3ζ intracellular T cell signaling sequence comprises, consists of, or consists essentially of the amino acid sequence of SEQ ID NO: 9.

The CARs described herein may be prepared in constructs with, e.g., self-cleaving peptides, such that the CAR constructs are bicistronic, tricistronic, etc. with anti-CD19, CD22, TSLPR, CD123, FLT3 CARs, etc., where the separate CARs are released upon cleavage of the peptides.

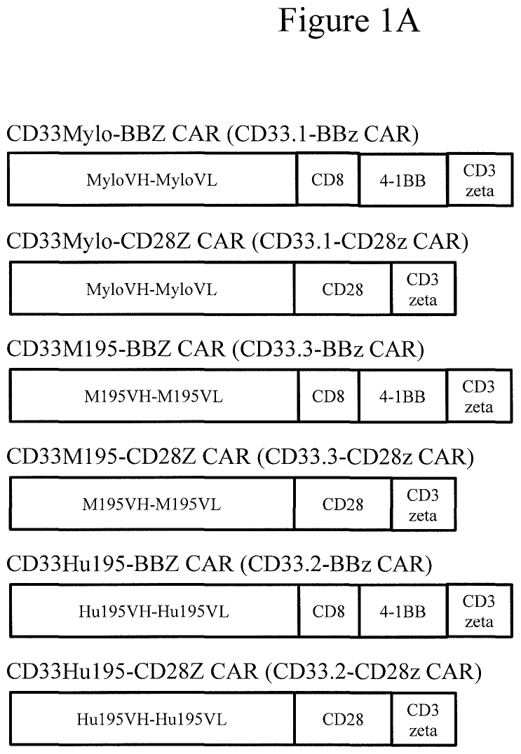

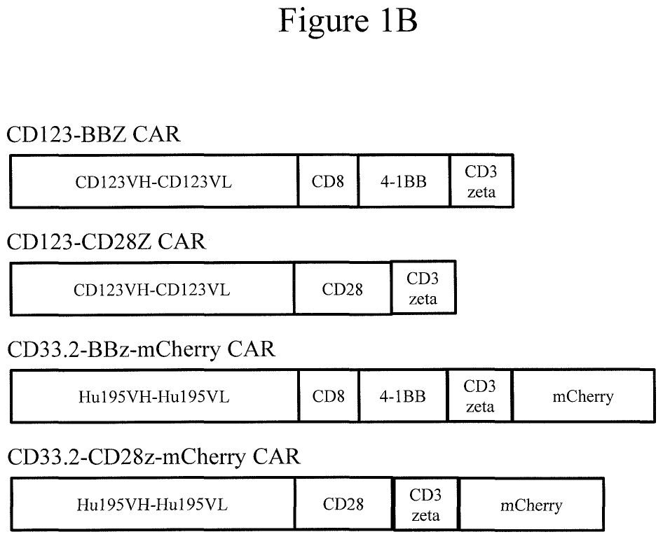

FIG. 1 presents schematic diagrams of exemplary CAR constructs, in accordance with embodiments of the invention.

Additional embodiments of the invention provide full-length CAR constructs comprising, consisting of, or consisting essentially of, any one or more of the amino acid sequences set forth in Tables 1-6 below.

TABLE 1
CD33Mylo-BBZ CAR

| | SEQ ID | | |
|---|---|---|---|
| Sequence | NO: | Segment | Notes |
| M | 1 | | start methionine |
| ALPVTALLLPLALLLHAARP | 2 | | signal peptide |
| EVQLVQSGAEVKKPGSSVK | 3 | Anti-CD33 scFv | heavy chain |
| VSCKASGYTITDSNIHWVRQ | | | |
| APGQSLEWIGYIYPYNGGTD | | | |
| YNQKFKNRATLTVDNPTNT | | | |
| AYMELSSLRSEDTAFYYCVN | | | |
| GNPWLAYWGQGTLVTVSS | | | |
| GGGGSGGGGSGGGGS | 4 | Anti-CD33 scFv | linker |
| DIQLTQSPSTLSASVGDRVTI | 5 | Anti-CD33 scFv | light chain |
| TCRASESLDNYGIRFLTWFQ | | | |
| QKPGKAPKLLMYAASNQGS | | | |
| GVPSRFSGSGSGTEFTLTISSL | | | |
| QPDDFATYYCQQTKEVPWS | | | |
| FGQGTKVEVKR | | | |
| TSSG | 38 | | AA added due to cloning |
| TTTPAPRPPTPAPTIASQPLSL | 6 | CD8 | CD8alpha hinge |
| RPEACRPAAGGAVHTRGLD | | | |
| FACD | | | |
| IYIWAPLAGTCGVLLLSLVIT | 7 | CD8 | CD8alpha transmembrane |
| LYC | | | domain |
| KRGRKKLLYIFKQPFMRPVQ | 8 | 4-1BB | intracellular domain |
| TTQEEDGCSCRFPEEEEGGC | | | |
| EL | | | |
| RVKFSRSADAPAYKQGQNQ | 9 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR | | | |

TABLE 2
CD33Mylo-CD28Z CAR

| | SEQ ID | | |
|---|---|---|---|
| Sequence | NO: | Segment | Notes |
| M | 1 | | start methionine |
| ALPVTALLLPLALLLHAARP | 2 | | signal peptide |
| EVQLVQSGAEVKKPGSSVK | 3 | Anti-CD33 scFv | heavy chain |
| VSCKASGYTITDSNIHWVRQ | | | |
| APGQSLEWIGYIYPYNGGTD | | | |
| YNQKFKNRATLTVDNPTNT | | | |
| AYMELSSLRSEDTAFYYCVN | | | |
| GNPWLAYWGQGTLVTVSS | | | |
| GGGGSGGGGSGGGGS | 4 | Anti-CD33 scFv | linker |
| DIQLTQSPSTLSASVGDRVTI | 5 | Anti-CD33 scFv | light chain |
| TCRASESLDNYGIRFLTWFQ | | | |
| QKPGKAPKLLMYAASNQGS | | | |
| GVPSRFSGSGSGTEFTLTISSL | | | |
| QPDDFATYYCQQTKEVPWS | | | |
| FGQGTKVEVKR | | | |
| AAAIEVMYPPPYLDNEKSNG | 10 | CD28 | CD28 hinge |
| TIIHVKGKHLCPSPLFPGPSK | | | |
| P | | | |
| FWVLVVVGGVLACYSLLVT | 11 | CD28 | CD28 transmembrane |
| VAFIIFWVRSKRSRLLHSDY | | | domain |
| MNMTPRRPGPTRKHYQPYA | | | |
| PPRDFAAYRS | | | |
| RVKFSRSADAPAYKQGQNQ | 9 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR | | | |

TABLE 3
CD33M195-BBZ CAR

| | SEQ ID | | |
|---|---|---|---|
| Sequence | NO: | Segment | Notes |
| M | 1 | | start methionine |
| ALPVTALLLPLALLLHAARP | 12 | | signal peptide |
| MALPVTALLLPLALLLHAAR | | | |
| P | | | |
| QVQLVQSGAEVKKPGSSVK | 13 | Anti-CD33 scFv | heavy chain |
| VSCKASGYTFTDYNMHWVR | | | |
| QAPGQGLEWIGYIYPYNGGT | | | |
| GYNQKFKSKATITADESTNT | | | |
| AYMELSSLRSEDTAVYYCA | | | |
| RGRPAMDYWGQGTLVTVSS | | | |
| GGGGSGGGGSGGGGS | 4 | Anti-CD33 scFv | linker |
| DIQMTQSPSSLSASVGDRVTI | 14 | Anti-CD33 scFv | light chain |
| TCRASESVDNYGISFMNWFQ | | | |
| QKPGKAPKLLIYAASNQGSG | | | |
| VPSRFSGSGSGTDFTLNISSL | | | |
| QPDDFATYYCQQSKEVPWT | | | |
| FGQGTKVEIK | | | |
| TSSG | 38 | | AA added due to cloning |
| TTTPAPRPPTPAPTIASQPLSL | 6 | CD8 | CD8alpha hinge |
| RPEACRPAAGGAVHTRGLD | | | |
| FACD | | | |
| IYIWAPLAGTCGVLLLSLVIT | 7 | CD8 | CD8alpha transmembrane |
| LYC | | | domain |
| KRGRKKLLYIFKQPFMRPVQ | 8 | 4-1BB | intracellular domain |
| TTQEEDGCSCRFPEEEEGGC | | | |
| EL | | | |
| RVKFSRSADAPAYKQGQNQ | 9 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR | | | |

TABLE 4
CD33M195-CD28Z CAR

| | SEQ ID | | |
|---|---|---|---|
| Sequence | NO: | Segment | Notes |
| M | 1 | | start methionine |
| ALPVTALLLPLALLLHAARP | 12 | | signal peptide |
| MALPVTALLLPLALLLHAAR | | | |
| P | | | |
| QVQLVQSGAEVKKPGSSVK | 13 | Anti-CD33 scFv | heavy chain |
| VSCKASGYTFTDYNMHWVR | | | |
| QAPGQGLEWIGYIYPYNGGT | | | |
| GYNQKFKSKATITADESTNT | | | |
| AYMELSSLRSEDTAVYYCA | | | |
| RGRPAMDYWGQGTLVTVSS | | | |
| GGGGSGGGGSGGGGS | 4 | Anti-CD33 scFv | linker |
| DIQMTQSPSSLSASVGDRVTI | 14 | Anti-CD33 scFv | light chain |
| TCRASESVDNYGISFMNWFQ | | | |
| QKPGKAPKLLIYAASNQGSG | | | |
| VPSRFSGSGSGTDFTLNISSL | | | |
| QPDDFATYYCQQSKEVPWT | | | |
| FGQGTKVEIK | | | |
| TSSG | 38 | | AA added due to cloning |
| AAAIEVMYPPPYLDNEKSNG | 10 | CD28 | CD28 hinge |
| TIIHVKGKHLCPSPLFPGPSK | | | |
| P | | | |
| FWVLVVVGGVLACYSLLVT | 11 | CD28 | CD28 transmembrane |
| VAFIIFWVRSKRSRLLHSDY | | | domain |
| MNMTPRRPGPTRKHYQPYA | | | |
| PPRDFAAYRS | | | |
| RVKFSRSADAPAYKQGQNQ | 9 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR | | | |

TABLE 5
CD33Hu195-BBZ CAR

| | SEQ ID | | |
|---|---|---|---|
| Sequence | NO: | Segment | Notes |
| M | 1 | | start methionine |
| ALPVTALLLPLALLLHAARP | 2 | | signal peptide |
| QVQLVQSGAEVKKPGSSVK | 15 | Anti-CD33 scFv | heavy chain |
| VSCKASGYTFTDYNMHWVR | | | |
| QAPGQGLEWIGYIYPYNGGT | | | |
| GYNQKFKSKATITADESTNT | | | |
| AYMELSSLRSEDTAVYYCA | | | |
| RGRPAMDYWGQGTLVTVSS | | | |
| GGGGSGGGGSGGGGS | 4 | Anti-CD33 scFv | linker |
| DIQMTQSPSSLSASVGDRVTI | 16 | Anti-CD33 scFv | light chain |
| TCRASESVDNYGISFMNWFQ | | | |
| QKPGKAPKLLIYAASNQGSG | | | |
| VPSRFSGSGSGTDFTLTISSL | | | |
| QPDDFATYYCQQSKEVPWT | | | |
| FGQGTKVEIK | | | |
| SG | 39 | | AA added due to cloning |
| TTTPAPRPPTPAPTIASQPLSL | 6 | CD8 | CD8alpha hinge |
| RPEACRPAAGGAVHTRGLD | | | |
| FACD | | | |
| IYIWAPLAGTCGVLLLSLVIT | 7 | CD8 | CD8alpha transmembrane |
| LYC | | | domain |
| KRGRKKLLYIFKQPFMRPVQ | 8 | 4-1BB | intracellular domain |
| TTQEEDGCSCRFPEEEEGGC | | | |
| EL | | | |
| RVKFSRSADAPAYKQGQNQ | 9 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR | | | |

TABLE 6
CD33Hu195-CD28Z CAR

| | SEQ ID | | |
|---|---|---|---|
| Sequence | NO: | Segment | Notes |
| M | 1 | | start methionine |
| ALPVTALLLPLALLLHAARP | 2 | | signal peptide |
| QVQLVQSGAEVKKPGSSVK | 15 | Anti-CD33 scFv | heavy chain |
| VSCKASGYTFTDYNMHWVR | | | |
| QAPGQGLEWIGYIYPYNGGT | | | |
| GYNQKFKSKATITADESTNT | | | |
| AYMELSSLRSEDTAVYYCA | | | |
| RGRPAMDYWGQGTLVTVSS | | | |
| GGGGSGGGGSGGGGS | 4 | Anti-CD33 scFv | linker |
| DIQMTQSPSSLSASVGDRVTI | 16 | Anti-CD33 scFv | light chain |
| TCRASESVDNYGISFMNWFQ | | | |
| QKPGKAPKLLIYAASNQGSG | | | |
| VPSRFSGSGSGTDFTLTISSL | | | |
| QPDDFATYYCQQSKEVPWT | | | |
| FGQGTKVEIK | | | |
| SG | 39 | | AA added due to cloning |
| AAAIEVMYPPPYLDNEKSNG | 10 | CD28 | CD28 hinge |
| TIIHVKGKHLCPSPLFPGPSK | | | |
| P | | | |
| FWVLVVVGGVLACYSLLVT | 11 | CD28 | CD28 transmembrane |
| VAFIIFWVRSKRSRLLHSDY | | | domain |
| MNMTPRRPGPTRKHYQPYA | | | |
| PPRDFAAYRS | | | |
| RVKFSRSADAPAYKQGQNQ | 9 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR | | | |

The CDR sequences are shown below in bold underlining.

Hu195 and M195:
(SEQ ID NO: 35)
QVQLVQSGAEVKKPGSSVKVSCKASGYTFTDYNMHWVRQAPGQGLEWIGY
IYPYNGGTGYNCIKFKSKATITADESTNTAYMELSSLRSEDTAVYYCARG
RPAMDYWGQGTLVTVSSGGGGSGGGGSGGGGSDIQMTQSPSSLSASVGDR
VTITCRASESVDNYGISFMNWFQQKPGKAPKLLIYAASNQGSGVPSRFSG
SGSGTDFTL [symbol] ISSLQPDDFATYYCQQSKEVPWTFGQGTKVEIKTSSG
where the boxed T of Hu195 is N for M195
CDRs:
(SEQ ID NO: 41)
DYNMH
(SEQ ID NO: 42)
YIYPYNGGTGYNQKFKSKA
(SEQ ID NO: 43)
GRPAMDYWGQ
(SEQ ID NO: 44)
RASESVDNYGISFMN
(SEQ ID NO: 45)
AASNQGS
(SEQ ID NO: 46)
QQSKEVPWT
Mylo:
(SEQ ID NO: 36)
EVQLVQSGAEVKKPGSSVKVSCKASGYTITDSNIHWVRQAPGQSLEWIGY
IYPYNGGTDYNQKFKNRATLTVDNPTNTAYMELSSLRSEDTAFYYCVNGN
PWLAYWGQGTLVTVSSGGGGSGGGGSGGGGSDIQLTQSPSTLSASVGDRV
TITCRASESLDNYGIRFLTWFQQKPGKAPKLLMYAASNQGSGVPSRFSGS
GSGTEFTLTISSLQPDDFATYYCQQTKEVPWSFGQGTKVEVKR
CDRs:
(SEQ ID NO: 47)
GYTITDSN
(SEQ ID NO: 48)
IYPYNGGT
(SEQ ID NO: 49)
VNGNPWLAY
(SEQ ID NO: 50)
ESLDNYGIRF
(SEQ ID NO: 51)
AAS
(SEQ ID NO: 52)
QQTKEVPWS

In an embodiment, the CAR construct (herein denoted CD33Mylo-BBZ) has the sequence:

(SEQ ID NO: 18)
MALPVTALLLPLALLLHAARPEVQLVQSGAEVKKPGSSVKVSCKASGYTI
TDSNIHWVRQAPGQSLEWIGYIYPYNGGTDYNQKFKNRATLTVDNPTNTA
YMELSSLRSEDTAFYYCVNGNPWLAYWGQGTLVTVSSGGGGSGGGGSGGG
GSDIQLTQSPSTLSASVGDRVTITCRASESLDNYGIRFLTWFQQKPGKAP
KLLMYAASNQGSGVPSRFSGSGSGTEFTLTISSLQPDDFATYYCQQTKEV
PWSFGQGTKVEVKRTSSGTTTPAPRPPTPAPTIASQPLSLRPEACRPAAG
GAVHTRGLDFACDIYIWAPLAGTCGVLLLSLVITLYCKRGRKKLLYIFKQ
PFMRPVQTTQEEDGCSCRFPEEEEGGCELRVKFSRSADAPAYKQGQNQLY
NELNLGRREEYDVLDKRRGRDPEMGGKPRRKNPQEGLYNELQKDKMAEAY
SEIGMKGERRRGKGHDGLYQGLSTATKDTYDALHMQALPPR.

In an embodiment, the CAR construct (herein denoted CD33Mylo-CD28Z) has the sequence:

(SEQ ID NO: 19)
MALPVTALLLPLALLLHAARPEVQLVQSGAEVKKPGSSVKVSCKASGYTI
TDSNIHWVRQAPGQSLEWIGYIYPYNGGTDYNQKFKNRATLTVDNPTNTA
YMELSSLRSEDTAFYYCVNGNPWLAYWGQGTLVTVSSGGGGSGGGGSGGG
GSDIQLTQSPSTLSASVGDRVTITCRASESLDNYGIRFLTWFQQKPGKAP
KLLMYAASNQGSGVPSRFSGSGSGTEFTLTISSLQPDDFATYYCQQTKEV
PWSFGQGTKVEVKRAAAIEVMYPPPYLDNEKSNGTIIHVKGKHLCPSPLF
PGPSKPFWVLVVVGGVLACYSLLVTVAFIIFWVRSKRSRLLHSDYMNMTP
RRPGPTRKHYQPYAPPRDFAAYRSRVKFSRSADAPAYKQGQNQLYNELNL
GRREEYDVLDKRRGRDPEMOGKPRRKNPQEGLYNELQKDKMAEAYSEIGM
KGERRRGKGHDGLYQGLSTATKDTYDALHMQALPPR.

In an embodiment, the CAR construct (herein denoted CD33M195-BBZ) has the sequence:

(SEQ ID NO: 20)
MALPVTALLLPLALLLHAARPMALPVTALLLPLALLLHAARPQVQLVQSG
AEVKKPGSSVKVSCKASGYTFTDYNMHWVRQAPGQGLEWIGYIYPYNGGT
GYNQKFKSKATITADESTNTAYMELSSLRSEDTAVYYCARGRPAMDYWGQ
GTLVTVSSGGGGSGGGGSGGGGSDIQMTQSPSSLSASVGDRVTITCRASE
SVDNYGISFMNWFQQKPGKAPKLLIYAASNQGSGVPSRFSGSGSGTDFTL
NISSLQPDDFATYYCQQSKEVPWTFGQGTKVEIKTSSGTTTPAPRPPTPA
PTIASQPLSLRPEACRPAAGGAVHTRGLDFACDIYIWAPLAGTCGVLLLS
LVITLYCKRGRKKLLYIFKQPFMRPVQTTQEEDGCSCRFPEEEEGGCELR
VKFSRSADAPAYKQGQNQLYNELNLGRREEYDVLDKRRGRDPEMGGKPRR
KNPQEGLYNELQKDKMAEAYSEIGMKGERRRGKGHDGLYQGLSTATKDTY
DALHMQALPPR.

In an embodiment, the CAR construct (herein denoted CD33M195-CD28Z) has the sequence:

(SEQ ID NO: 21)
MALPVTALLLPLALLLHAARPMALPVTALLLPLALLLHAARPQVQLVQSG
AEVKKPGSSVKVSCKASGYTFTDYNMHWVRQAPGQGLEWIGYIYPYNGGT
GYNQKFKSKATITADESTNTAYMELSSLRSEDTAVYYCARGRPAMDYWGQ
GTLVTVSSGGGGSGGGGSGGGGSDIQMTQSPSSLSASVGDRVTITCRASE
SVDNYGISFMNWFQQKPGKAPKLLIYAASNQGSGVPSRFSGSGSGTDFTL
NISSLQPDDFATYYCQQSKEVPWTFGQGTKVEIKTSSGAAAIEVMYPPPY
LDNEKSNGTIIHVKGKHLCPSPLFPGPSKPFWVLVVVGGVLACYSLLVTV
AFIIFWVRSKRSRLLHSDYMNMTPRRPGPTRKHYQPYAPPRDFAAYRSRV
KFSRSADAPAYKQGQNQLYNELNLGRREEYDVLDKRRGRDPEMGGKPRRK
NPQEGLYNELQKDKMAEAYSEIGMKGERRRGKGHDGLYQGLSTATKDTYD
ALHMQALPPR.

In an embodiment, the CAR construct (herein denoted CD33Hu195-BBZ) has the sequence:

(SEQ ID NO: 22)
MALPVTALLLPLALLLHAARPQVQLVQSGAEVKKPGSSVKVSCKASGYTF
TDYNMHWVRQAPGQGLEWIGYIYPYNGGTGYNQKFKSKATITADESTNTA
YMELSSLRSEDTAVYYCARGRPAMDYWGQGTLVTVSSGGGGSGGGGSGGG
GSDIQMTQSPSSLSASVGDRVTITCRASESVDNYGISFMNWFQQKPGKAP
KLLIYAASNQGSGVPSRFSGSGSGTDFTLTISSLQPDDFATYYCQQSKEV
PWTFGQGTKVEIKSGTTTPAPRPPTPAPTIASQPLSLRPEACRPAAGGAV
HTRGLDFACDIYIWAPLAGTCGVLLLSLVITLYCKRGRKKLLYIFKQPFM
RPVQTTQEEDGCSCRFPEEEEGGCELRVKFSRSADAPAYKQGQNQLYNEL
NLGRREEYDVLDKRRGRDPEMGGKPRRKNPQEGLYNELQKDKMAEAYSEI
GMKGERRRGKGHDGLYQGLSTATKDTYDALHMQALPPR.

In an embodiment, the CAR construct (herein denoted CD33Hu195-CD28Z) has the sequence:

(SEQ ID NO: 23)
MALPVTALLLPLALLLHAARPQVQLVQSGAEVKKPGSSVKVSCKASGYT
FTDYNMHWVRQAPGQGLEWIGYIYPYNGGTGYNQKFKSKATITADESTN
TAYMELSSLRSEDTAVYYCARGRPAMDYWGQGTLVTVSSGGGGSGGGGS
GGGGSDIQMTQSPSSLSASVGDRVTITCRASESVDNYGISFMNWFQQKP
GKAPKLLIYAASNQGSGVPSRFSGSGSGTDFTLTISSLQPDDFATYYCQ
QSKEVPWTFGQGTKVEIKSGAAAIEVMYPPPYLDNEKSNGTIIHVKGKH
LCPSPLFPGPSKPFWVLVVVGGVLACYSLLVTVAFIIFWVRSKRSRLLH
SDYMNMTPRRPGPTRKHYQPYAPPRDFAAYRSRVKFSRSADAPAYQQGQ
NQLYNELNLGRREEYDVLDKRRGRDPEMGGKPRRKNPQEGLYNELQKDK
MAEAYSEIGMKGERRRGKGHDGLYQGLSTATKDTYDALHMQALPPR.
(SEQ ID NO: 37)
The Q in RVKFSRSADAPAYQ

may be substituted with K.

The sequence of GS may be at the C-terminus of any of the CARs described herein.

Included in the scope of the invention are functional portions of the inventive CAR constructs described herein. The term “functional portion” when used in reference to a CAR refers to any part or fragment of the CAR constructs of the invention, which part or fragment retains the biological activity of the CAR construct of which it is a part (the parent CAR construct). Functional portions encompass, for example, those parts of a CAR construct that retain the ability to recognize target cells, or detect, treat, or prevent cancer, to a similar extent, the same extent, or to a higher extent, as the parent CAR construct. In reference to the parent CAR construct, the functional portion can comprise, for instance, about 10%, about 25%, about 30%, about 50%, about 68%, about 80%, about 90%, about 95%, or more, of the parent CAR.

The functional portion can comprise additional amino acids at the amino or carboxy terminus of the portion, or at both termini, which additional amino acids are not found in the amino acid sequence of the parent CAR construct. Desirably, the additional amino acids do not interfere with the biological function of the functional portion, e.g., recognize target cells, detect cancer, treat or prevent cancer, etc. More desirably, the additional amino acids enhance the biological activity as compared to the biological activity of the parent CAR construct.

Included in the scope of the invention are functional variants of the inventive CAR constructs described herein. The term “functional variant,” as used herein, refers to a CAR construct, polypeptide, or protein having substantial or significant sequence identity or similarity to a parent CAR construct, which functional variant retains the biological activity of the CAR of which it is a variant. Functional variants encompass, for example, those variants of the CAR construct described herein (the parent CAR construct) that retain the ability to recognize target cells to a similar extent, the same extent, or to a higher extent, as the parent CAR construct. In reference to the parent CAR construct, the functional variant can, for instance, be at least about 30%, about 50%, about 75%, about 80%, about 90%, about 91%, about 92%, about 93%, about 94%, about 95%, about 96%, about 97%, about 98%, about 99% or more identical in amino acid sequence to the parent CAR construct.

A functional variant can, for example, comprise the amino acid sequence of the parent CAR with at least one conservative amino acid substitution. Alternatively or additionally, the functional variants can comprise the amino acid sequence of the parent CAR construct with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR construct.

Amino acid substitutions of the inventive CAR constructs are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The CAR construct can consist essentially of the specified amino acid sequence or sequences described herein, such that other components, e.g., other amino acids, do not materially change the biological activity of the functional variant.

The CAR constructs of embodiments of the invention (including functional portions and functional variants) can be of any length, i.e., can comprise any number of amino acids, provided that the CAR constructs (or functional portions or functional variants thereof) retain their biological activity, e.g., the ability to specifically bind to antigen, detect diseased cells in a mammal, or treat or prevent disease in a mammal, etc. For example, the CAR can be about 50 to about 5000 amino acids long, such as 50, 70, 75, 100, 125, 150, 175, 200, 300, 400, 500, 600, 700, 800, 900, 1000 or more amino acids in length.

The CAR constructs of embodiments of the invention (including functional portions and functional variants of the invention) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The CAR constructs of embodiments of the invention (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The CAR constructs of embodiments of the invention (including functional portions and functional variants thereof) can be obtained by methods known in the art. The CAR constructs may be made by any suitable method of making polypeptides or proteins, including de novo synthesis. Also, the CAR constructs can be recombinantly produced using the nucleic acids described herein using standard recombinant methods. See, for instance, Green et al., Molecular Cloning: A Laboratory Manual, 4th ed., Cold Spring Harbor Press, Cold Spring Harbor, N.Y. 2012. Further, portions of some of the CAR constructs of the invention (including functional portions and functional variants thereof) can be isolated and/or purified from a source, such as a plant, a bacterium, an insect, a mammal, e.g., a rat, a human, etc. Methods of isolation and purification are well-known in the art. Alternatively, the CAR constructs described herein (including functional portions and functional variants thereof) can be commercially synthesized by companies, such as Synpep (Dublin, Calif.), Peptide Technologies Corp. (Gaithersburg, Md.), and Multiple Peptide Systems (San Diego, Calif.). In this respect, the inventive CAR constructs can be synthetic, recombinant, isolated, and/or purified.

Further provided by an embodiment of the invention is a nucleic acid comprising a nucleotide sequence encoding any of the CAR constructs described herein (including functional portions and functional variants thereof). The nucleic acids of the invention may comprise a nucleotide sequence encoding any of the leader sequences, antigen binding domains, transmembrane domains, linkers, and/or intracellular T cell signaling domains described herein.

In an embodiment, the nucleic acid comprises a nucleotide sequence that encodes any CAR construct described herein. In an embodiment of the invention, the nucleic acid may comprise, consist of, or consist essentially of, the nucleotide sequence of any of the following.

CD33My10-BBZ CAR
(SEQ ID NO: 24)
ATGGCCCTGCCTGTGACAGCCCTGCTGCTGCCCCTGGCTCTGCTGCTGCATGCCGCC
AGACCTGAGGTGCAGCTGGTGCAGTCTGGCGCCGAAGTGAAGAAACCCGGCAGCAG
CGTGAAGGTGTCCTGCAAGGCCAGCGGCTACACCATCACCGACAGCAACATCCACT
GGGTGCGCCAGGCCCCTGGCCAGAGCCTGGAATGGATCGGCTACATCTACCCCTAC
AACGGCGGCACCGACTACAACCAGAAGTTCAAGAACCGGGCCACCCTGACCGTGGA
CAACCCCACCAACACCGCCTACATGGAACTGAGCAGCCTGCGGAGCGAGGACACCG
CCTTCTACTACTGCGTGAACGGCAACCCCTGGCTGGCCTACTGGGGCCAGGGAACCC
TGGTGACAGTGTCTAGCGGCGGAGGCGGATCTGGAGGGGGAGGATCTGGCGGCGGA
GGAAGCGACATCCAGCTGACCCAGAGCCCCAGCACCCTGAGCGCCAGCGTGGGCGA
CAGAGTGACCATCACCTGTCGGGCCAGCGAGAGCCTGGACAACTACGGCATCCGGT
TTCTGACCTGGTTCCAGCAGAAGCCCGGCAAGGCCCCCAAGCTGCTGATGTACGCCG
CCAGCAATCAGGGCAGCGGCGTGCCCAGCAGATTCAGCGGCTCTGGCAGCGGAACC
GAGTTCACCCTGACCATCAGCAGCCTGCAGCCCGACGACTTCGCCACCTACTACTGC
CAGCAGACCAAAGAGGTGCCCTGGTCCTTCGGCCAGGGCACCAAGGTGGAAGTGAA
GCGGACTAGTTCCGGAACCACGACGCCAGCGCCGCGACCACCAACACCGGCGCCCA
CCATCGCGTCGCAGCCCCTGTCCCTGCGCCCAGAGGCGTGCCGGCCAGCGGCGGGG
GGCGCAGTGCACACGAGGGGGCTGGACTTCGCCTGTGATATCTACATCTGGGCGCC
CTTGGCCGGGACTTGTGGGGTCCTTCTCCTGTCACTGGTTATCACCCTTTACTGCAAA
CGGGGCAGAAAGAAACTCCTGTATATATTCAAACAACCATTTATGAGACCAGTACA
AACTACTCAAGAGGAAGATGGCTGTAGCTGCCGATTTCCAGAAGAAGAAGAAGGAG
GATGTGAACTGAGAGTGAAGTTCAGCAGGAGCGCAGACGCCCCCGCGTACAAGCAG
GGCCAGAACCAGCTCTATAACGAGCTCAATCTAGGACGAAGAGAGGAGTACGATGT
TTTGGACAAGAGACGTGGCCGGGACCCTGAGATGGGGGGAAAGCCGAGAAGGAAG
AACCCTCAGGAAGGCCTGTACAATGAACTGCAGAAAGATAAGATGGCGGAGGCCTA
CAGTGAGATTGGGATGAAAGGCGAGCGCCGGAGGGGCAAGGGGCACGATGGCCTTT
ACCAGGGTCTCAGTACAGCCACCAAGGACACCTACGACGCCCTTCACATGCAGGCC
CTGCCCCCTCGCTAA
CD33My10-CD28Z CAR
(SEQ ID NO: 25)
ATGGCCCTGCCTGTGACAGCCCTGCTGCTGCCCCTGGCTCTGCTGCTGCATGCCGCC
AGACCTGAGGTGCAGCTGGTGCAGTCTGGCGCCGAAGTGAAGAAACCCGGCAGCAG
CGTGAAGGTGTCCTGCAAGGCCAGCGGCTACACCATCACCGACAGCAACATCCACT
GGGTGCGCCAGGCCCCTGGCCAGAGCCTGGAATGGATCGGCTACATCTACCCCTAC
AACGGCGGCACCGACTACAACCAGAAGTTCAAGAACCGGGCCACCCTGACCGTGGA
CAACCCCACCAACACCGCCTACATGGAACTGAGCAGCCTGCGGAGCGAGGACACCG
CCTTCTACTACTGCGTGAACGGCAACCCCTGGCTGGCCTACTGGGGCCAGGGAACCC
TGGTGACAGTGTCTAGCGGCGGAGGCGGATCTGGAGGGGGAGGATCTGGCGGCGGA
GGAAGCGACATCCAGCTGACCCAGAGCCCCAGCACCCTGAGCGCCAGCGTGGGCGA
CAGAGTGACCATCACCTGTCGGGCCAGCGAGAGCCTGGACAACTACGGCATCCGGT
TTCTGACCTGGTTCCAGCAGAAGCCCGGCAAGGCCCCCAAGCTGCTGATGTACGCCG
CCAGCAATCAGGGCAGCGGCGTGCCCAGCAGATTCAGCGGCTCTGGCAGCGGAACC
GAGTTCACCCTGACCATCAGCAGCCTGCAGCCCGACGACTTCGCCACCTACTACTGC
CAGCAGACCAAAGAGGTGCCCTGGTCCTTCGGCCAGGGCACCAAGGTGGAAGTGAA
GCGGACTAGTTCCGGAGCCGCCGCCATCGAAGTGATGTACCCCCCTCCCTACCTGGA
TAACGAGAAGAGCAACGGCACCATCATCCACGTGAAGGGAAAGCACCTGTGTCCCA
GCCCCCTGTTTCCCGGCCCTAGCAAGCCCTTCTGGGTGCTGGTGGTGGTCGGCGGAG
TGCTGGCCTGCTACAGCCTCCTGGTGACCGTGGCCTTCATCATCTTCTGGGTGAGGA
GCAAGAGGTCCAGGCTGCTGCACAGCGACTACATGAATATGACCCCCAGAAGGCCC
GGCCCCACCAGAAAGCACTATCAGCCCTACGCCCCCCCCAGGGACTTTGCCGCCTAC
AGGAGCAGGGTGAAGTTCAGCAGATCCGCCGATGCCCCTGCTTACCAGCAGGGCCA
GAACCAGCTGTATAACGAGCTGAACCTGGGCAGGAGGGAGGAATACGACGTGCTGG
ATAAGAGGAGGGGAAGGGACCCCGAGATGGGCGGAAAGCCCAGGAGGAAGAACCC
CCAGGAGGGCCTGTACAATGAGCTGCAGAAAGACAAGATGGCCGAGGCCTACAGC
GAGATCGGCATGAAGGGCGAGAGGAGGAGGGGCAAGGGCCATGACGGCCTGTACC
AAGGCCTGTCCACCGCCACCAAGGATACCTACGACGCCCTGCACATGCAGGCCCTG
CCTCCCAGGGGATCCTAA
CD33M195-BBZ CAR
(SEQ ID NO: 26)
ATGGCCCTGCCTGTGACAGCCCTGCTGCTGCCCCTGGCTCTGCTGCTGCATGCCGCC
AGACCTATGGCTCTGCCCGTGACCGCTCTCCTCCTGCCACTGGCACTGCTCCTCCAC
GCTGCTAGACCCCAGGTGCAGCTGGTGCAGTCTGGCGCCGAAGTGAAGAAACCCGG
CAGCAGCGTGAAGGTGTCCTGCAAGGCCAGCGGCTACACCTTCACCGACTACAACA
TGCACTGGGTGCGCCAGGCTCCAGGCCAGGGACTGGAATGGATCGGCTACATCTAC
CCCTACAACGGCGGCACCGGCTACAACCAGAAGTTCAAGAGCAAGGCCACCATCAC
CGCCGACGAGAGCACCAACACCGCCTACATGGAACTGAGCAGCCTGCGGAGCGAGG
ACACCGCCGTGTACTACTGCGCCAGAGGCAGACCCGCCATGGACTACTGGGGCCAG
GGCACCCTGGTGACAGTGTCTAGCGGAGGCGGAGGCTCTGGCGGCGGAGGAAGTGG
CGGAGGCGGCAGCGATATCCAGATGACCCAGAGCCCCAGCAGCCTGAGCGCCAGCG
TGGGCGACAGAGTGACCATCACCTGTCGGGCCAGCGAGAGCGTGGACAACTACGGC
ATCAGCTTCATGAACTGGTTCCAGCAGAAGCCCGGCAAGGCCCCCAAGCTGCTGAT
CTACGCCGCCAGCAATCAGGGCAGCGGCGTGCCCAGCAGATTCAGCGGCTCTGGCA
GCGGCACCGACTTCACCCTGAACATCAGCAGCCTGCAGCCCGACGACTTCGCCACCT
ACTACTGCCAGCAGAGCAAAGAGGTGCCCTGGACCTTCGGACAGGGCACCAAGGTG
GAAATCAAGACTAGTTCCGGAACCACGACGCCAGCGCCGCGACCACCAACACCGGC
GCCCACCATCGCGTCGCAGCCCCTGTCCCTGCGCCCAGAGGCGTGCCGGCCAGCGG
CGGGGGGCGCAGTGCACACGAGGGGGCTGGACTTCGCCTGTGATATCTACATCTGG
GCGCCCTTGGCCGGGACTTGTGGGGTCCTTCTCCTGTCACTGGTTATCACCCTTTACT
GCAAACGGGGCAGAAAGAAACTCCTGTATATATTCAAACAACCATTTATGAGACCA
GTACAAACTACTCAAGAGGAAGATGGCTGTAGCTGCCGATTTCCAGAAGAAGAAGA
AGGAGGATGTGAACTGAGAGTGAAGTTCAGCAGGAGCGCAGACGCCCCCGCGTACA
AGCAGGGCCAGAACCAGCTCTATAACGAGCTCAATCTAGGACGAAGAGAGGAGTAC
GATGTTTTGGACAAGAGACGTGGCCGGGACCCTGAGATGGGGGGAAAGCCGAGAA
GGAAGAACCCTCAGGAAGGCCTGTACAATGAACTGCAGAAAGATAAGATGGCGGA
GGCCTACAGTGAGATTGGGATGAAAGGCGAGCGCCGGAGGGGCAAGGGGCACGAT
GGCCTTTACCAGGGTCTCAGTACAGCCACCAAGGACACCTACGACGCCCTTCACATG
CAGGCCCTGCCCCCTCGCTAA
CD33M195-CD28Z CAR
(SEQ ID NO: 27)
ATGGCCCTGCCTGTGACAGCCCTGCTGCTGCCCCTGGCTCTGCTGCTGCATGCCGCC
AGACCTATGGCTCTGCCCGTGACCGCTCTCCTCCTGCCACTGGCACTGCTCCTCCAC
GCTGCTAGACCCCAGGTGCAGCTGGTGCAGTCTGGCGCCGAAGTGAAGAAACCCGG
CAGCAGCGTGAAGGTGTCCTGCAAGGCCAGCGGCTACACCTTCACCGACTACAACA
TGCACTGGGTGCGCCAGGCTCCAGGCCAGGGACTGGAATGGATCGGCTACATCTAC
CCCTACAACGGCGGCACCGGCTACAACCAGAAGTTCAAGAGCAAGGCCACCATCAC
CGCCGACGAGAGCACCAACACCGCCTACATGGAACTGAGCAGCCTGCGGAGCGAGG
ACACCGCCGTGTACTACTGCGCCAGAGGCAGACCCGCCATGGACTACTGGGGCCAG
GGCACCCTGGTGACAGTGTCTAGCGGAGGCGGAGGCTCTGGCGGCGGAGGAAGTGG
CGGAGGCGGCAGCGATATCCAGATGACCCAGAGCCCCAGCAGCCTGAGCGCCAGCG
TGGGCGACAGAGTGACCATCACCTGTCGGGCCAGCGAGAGCGTGGACAACTACGGC
ATCAGCTTCATGAACTGGTTCCAGCAGAAGCCCGGCAAGGCCCCCAAGCTGCTGAT
CTACGCCGCCAGCAATCAGGGCAGCGGCGTGCCCAGCAGATTCAGCGGCTCTGGCA
GCGGCACCGACTTCACCCTGAACATCAGCAGCCTGCAGCCCGACGACTTCGCCACCT
ACTACTGCCAGCAGAGCAAAGAGGTGCCCTGGACCTTCGGACAGGGCACCAAGGTG
GAAATCAAGACTAGTTCCGGAGCCGCCGCCATCGAAGTGATGTACCCCCCTCCCTAC
CTGGATAACGAGAAGAGCAACGGCACCATCATCCACGTGAAGGGAAAGCACCTGTG
TCCCAGCCCCCTGTTTCCCGGCCCTAGCAAGCCCTTCTGGGTGCTGGTGGTGGTCGG
CGGAGTGCTGGCCTGCTACAGCCTCCTGGTGACCGTGGCCTTCATCATCTTCTGGGT
GAGGAGCAAGAGGTCCAGGCTGCTGCACAGCGACTACATGAATATGACCCCCAGAA
GGCCCGGCCCCACCAGAAAGCACTATCAGCCCTACGCCCCCCCCAGGGACTTTGCC
GCCTACAGGAGCAGGGTGAAGTTCAGCAGATCCGCCGATGCCCCTGCTTACCAGCA
GGGCCAGAACCAGCTGTATAACGAGCTGAACCTGGGCAGGAGGGAGGAATACGAC
GIGCTGGATAAGAGGAGGGGAAGGGACCCCGAGATGGGCGGAAAGCCCAGGAGGA
AGAACCCCCAGGAGGGCCTGTACAATGAGCTGCAGAAAGACAAGATGGCCGAGGC
CTACAGCGAGATCGGCATGAAGGGCGAGAGGAGGAGGGGCAAGGGCCATGACGGC
CTGTACCAAGGCCTGTCCACCGCCACCAAGGATACCTACGACGCCCTGCACATGCA
GGCCCTGCCTCCCAGGGGATCCTAA
CD33Hu195-BBZ CAR
(SEQ ID NO: 28)
ATGGCTCTGCCCGTCACAGCTCTGCTGCTGCCTCTGGCCCTGCTGCTGCACGCCGCC
AGACCTCAGGTGCAGCTCGTGCAGAGCGGCGCTGAGGTGAAGAAACCTGGCAGCAG
CGTGAAGGTGAGCTGCAAGGCCTCCGGCTACACCTTCACCGACTACAACATGCACT
GGGTGAGGCAAGCCCCTGGCCAGGGACTGGAGTGGATCGGCTACATCTACCCTTAC
AACGGCGGCACAGGCTACAACCAGAAGTTCAAGTCCAAGGCCACCATCACCGCCGA
TGAGTCCACCAATACCGCCTACATGGAGCTCAGCAGCCTGAGGTCCGAGGACACAG
CCGTCTACTACTGCGCCAGGGGCAGGCCCGCTATGGACTACTGGGGCCAGGGCACC
CTGGTGACAGTGAGCTCTGGTGGCGGCGGATCCGGCGGCGGCGGCAGCGGCGGCGG
CGGCTCCGACATTCAGATGACCCAGAGCCCTAGCAGCCTGAGCGCTTCCGTGGGAG
ACAGGGTGACCATCACATGCAGGGCCTCCGAGAGCGTGGACAATTACGGCATCAGC
TTCATGAACTGGTTCCAGCAGAAGCCCGGCAAGGCCCCCAAACTGCTGATCTATGCC
GCCAGCAATCAGGGCTCCGGCGTGCCTAGCAGGTTTTCCGGCAGCGGCAGCGGCAC
CGACTTTACCCTGACCATCTCCAGCCTGCAGCCTGACGATTTCGCCACCTACTACTG
CCAGCAGAGCAAGGAGGTGCCTTGGACCTTTGGACAGGGCACAAAGGTGGAGATCA
AGTCCGGAACCACGACGCCAGCGCCGCGACCACCAACACCGGCGCCCACCATCGCG
TCGCAGCCCCTGTCCCTGCGCCCAGAGGCGTGCCGGCCAGCGGCGGGGGGCGCAGT
GCACACGAGGGGGCTGGACTTCGCCTGTGATATCTACATCTGGGCGCCCTTGGCCGG
GACTTGTGGGGTCCTTCTCCTGTCACTGGTTATCACCCTTTACTGCAAACGGGGCAG
AAAGAAACTCCTGTATATATTCAAACAACCATTTATGAGACCAGTACAAACTACTCA
AGAGGAAGATGGCTGTAGCTGCCGATTTCCAGAAGAAGAAGAAGGAGGATGTGAA
CTGAGAGTGAAGTTCAGCAGGAGCGCAGACGCCCCCGCGTACAAGCAGGGCCAGA
ACCAGCTCTATAACGAGCTCAATCTAGGACGAAGAGAGGAGTACGATGTTTTGGAC
AAGAGACGTGGCCGGGACCCTGAGATGGGGGGAAAGCCGAGAAGGAAGAACCCTC
AGGAAGGCCTGTACAATGAACTGCAGAAAGATAAGATGGCGGAGGCCTACAGTGA
GATTGGGATGAAAGGCGAGCGCCGGAGGGGCAAGGGGCACGATGGCCTTTACCAG
GGTCTCAGTACAGCCACCAAGGACACCTACGACGCCCTTCACATGCAGGCCCTGCCC
CCTCGCTAA
CD33Hu195-CD28Z CAR
(SEQ ID NO: 29)
ATGGCTCTGCCCGTCACAGCTCTGCTGCTGCCTCTGGCCCTGCTGCTGCACGCCGCC
AGACCTCAGGTGCAGCTCGTGCAGAGCGGCGCTGAGGTGAAGAAACCTGGCAGCAG
CGTGAAGGTGAGCTGCAAGGCCTCCGGCTACACCTTCACCGACTACAACATGCACT
GGGTGAGGCAAGCCCCTGGCCAGGGACTGGAGTGGATCGGCTACATCTACCCTTAC
AACGGCGGCACAGGCTACAACCAGAAGTTCAAGTCCAAGGCCACCATCACCGCCGA
TGAGTCCACCAATACCGCCTACATGGAGCTCAGCAGCCTGAGGTCCGAGGACACAG
CCGTCTACTACTGCGCCAGGGGCAGGCCCGCTATGGACTACTGGGGCCAGGGCACC
CTGGTGACAGTGAGCTCTGGTGGCGGCGGATCCGGCGGCGGCGGCAGCGGCGGCGG
CGGCTCCGACATTCAGATGACCCAGAGCCCTAGCAGCCTGAGCGCTTCCGTGGGAG
ACAGGGTGACCATCACATGCAGGGCCTCCGAGAGCGTGGACAATTACGGCATCAGC
TTCATGAACTGGTTCCAGCAGAAGCCCGGCAAGGCCCCCAAACTGCTGATCTATGCC
GCCAGCAATCAGGGCTCCGGCGTGCCTAGCAGGTTTTCCGGCAGCGGCAGCGGCAC
CGACTTTACCCTGACCATCTCCAGCCTGCAGCCTGACGATTTCGCCACCTACTACTG
CCAGCAGAGCAAGGAGGTGCCTTGGACCTTTGGACAGGGCACAAAGGTGGAGATCA
AGTCCGGAGCCGCCGCCATCGAAGTGATGTACCCCCCTCCCTACCTGGATAACGAG
AAGAGCAACGGCACCATCATCCACGTGAAGGGAAAGCACCTGTGTCCCAGCCCCCT
GTTTCCCGGCCCTAGCAAGCCCTTCTGGGTGCTGGTGGTGGTCGGCGGAGTGCTGGC
CTGCTACAGCCTCCTGGTGACCGTGGCCTTCATCATCTTCTGGGTGAGGAGCAAGAG
GTCCAGGCTGCTGCACAGCGACTACATGAATATGACCCCCAGAAGGCCCGGCCCCA
CCAGAAAGCACTATCAGCCCTACGCCCCCCCCAGGGACTTTGCCGCCTACAGGAGC
AGGGTGAAGTTCAGCAGATCCGCCGATGCCCCTGCTTACCAGCAGGGCCAGAACCA
GCTGTATAACGAGCTGAACCTGGGCAGGAGGGAGGAATACGACGTGCTGGATAAGA
GGAGGGGAAGGGACCCCGAGATGGGCGGAAAGCCCAGGAGGAAGAACCCCCAGGA
GGGCCTGTACAATGAGCTGCAGAAAGACAAGATGGCCGAGGCCTACAGCGAGATCG
GCATGAAGGGCGAGAGGAGGAGGGGCAAGGGCCATGACGGCCTGTACCAAGGCCT
GTCCACCGCCACCAAGGATACCTACGACGCCCTGCACATGCAGGCCCTGCCTCCCAG
GGGATCCTAA

“Nucleic acid,” as used herein, includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some embodiments, the nucleic acid does not comprise any insertions, deletions, inversions, and/or substitutions. However, it may be suitable in some instances, as discussed herein, for the nucleic acid to comprise one or more insertions, deletions, inversions, and/or substitutions. In some embodiments, the nucleic acid may encode additional amino acid sequences that do not affect the function of the CAR construct and which may or may not be translated upon expression of the nucleic acid by a host cell.

In an embodiment, any nucleotide sequence herein may be codon-optimized. Without being bound to a particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency. In an embodiment of the invention, the codon-optimized nucleotide sequence may comprise, consist, or consist essentially of any one of the nucleic acid sequences described herein.

The nucleic acids of an embodiment of the invention may be recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

A recombinant nucleic acid may be one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques, such as those described in Green et al., supra. The nucleic acids can be constructed based on chemical synthesis and/or enzymatic ligation reactions using procedures known in the art. See, for example, Green et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

The nucleic acid can comprise any isolated or purified nucleotide sequence which encodes any of the CAR constructs or functional portions or functional variants thereof. Alternatively, the nucleotide sequence can comprise a nucleotide sequence which is degenerate to any of the sequences or a combination of degenerate sequences.

An embodiment of the invention also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive CAR constructs. It is generally appreciated that conditions can be rendered more stringent by the addition of increasing amounts of formamide.

The invention also provides a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein.

In an embodiment, the nucleic acids of the invention can be incorporated into a recombinant expression vector. In this regard, an embodiment of the invention provides recombinant expression vectors comprising any of the nucleic acids of the invention. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector. An exemplary vector backbone is the lenti-vector backbone of SEQ ID NO: 30.

In an embodiment, the recombinant expression vector of the invention can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-Cl, pMAM, and pMAMneo (Clontech). The recombinant expression vector may be a viral vector, e.g., a retroviral vector or a lentiviral vector.

In an embodiment, the recombinant expression vectors of the invention can be prepared using standard recombinant DNA techniques described in, for example, Green et al., supra. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

The recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. The recombinant expression vector may also comprise restriction sites to facilitate cloning.

The recombinant expression vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the CAR construct (including functional portions and functional variants thereof), or to the nucleotide sequence which is complementary to or which hybridizes to the nucleotide sequence encoding the CAR construct. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine daminase, purine nucleoside phosphorylase, and nitroreductase.

Included in the scope of the invention are conjugates, e.g., bioconjugates, comprising any of the inventive CAR constructs (including any of the functional portions or variants thereof), nucleic acids, recombinant expression vectors, host cells, or populations of host cells. Conjugates, as well as methods of synthesizing conjugates in general, are known in the art.

An embodiment of the invention further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5α cell. For purposes of producing a recombinant CAR construct, the host cell may be a mammalian cell. The host cell may be a human cell. While the host cell can be of any cell type, can originate from any type of tissue, and can be of any developmental stage, the host cell may be a peripheral blood lymphocyte (PBL) or a peripheral blood mononuclear cell (PBMC). The host cell may be a T cell or an NK cell.

For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4⁺/CD8⁺ double positive T cells, CD4⁺ helper T cells, e.g., Th₁ and Th₂ cells, CD8⁺ T cells (e.g., cytotoxic T cells), tumor infiltrating cells, memory T cells, naïve T cells, and the like. The T cell may be a CD8⁺ T cell or a CD4⁺ T cell.

Also provided by an embodiment of the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

The inventive CAR constructs (including functional portions and variants thereof), nucleic acids, recombinant expression vectors, and host cells (including populations thereof), all of which are collectively referred to as “inventive CAR construct materials” hereinafter, can be isolated and/or purified. The term “isolated,” as used herein, means having been removed from its natural environment. The term “purified” or “isolated” does not require absolute purity or isolation; rather, it is intended as a relative term. Thus, for example, a purified (or isolated) host cell preparation is one in which the host cell is more pure than cells in their natural environment within the body. Such host cells may be produced, for example, by standard purification techniques. In some embodiments, a preparation of a host cell is purified such that the host cell represents at least about 50%, for example at least about 70%, of the total cell content of the preparation. For example, the purity can be at least about 50%, can be greater than about 60%, about 70% or about 80%, or can be about 100%.

The inventive CAR construct materials can be formulated into a composition, such as a pharmaceutical composition. In this regard, an embodiment of the invention provides a pharmaceutical composition comprising any of the inventive CAR construct materials described herein and a pharmaceutically acceptable carrier. The inventive pharmaceutical compositions containing any of the inventive CAR construct materials can comprise more than one inventive CAR construct material, e.g., a CAR construct and a nucleic acid, or two or more different CAR constructs. Alternatively, the pharmaceutical composition can comprise an inventive CAR construct material in combination with other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In a preferred embodiment, the pharmaceutical composition comprises the inventive host cell or populations thereof.

With respect to pharmaceutical compositions, the pharamaceutically acceptable carrier can be any of those conventionally used and is limited only by chemico-physical considerations, such as solubility and lack of reactivity with the active agent(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which has no detrimental side effects or toxicity under the conditions of use.

The choice of carrier will be determined in part by the particular inventive CAR construct material, as well as by the particular method used to administer the inventive CAR construct material. Accordingly, there are a variety of suitable formulations of the pharmaceutical composition of the invention. Methods for preparing administrable (e.g., parenterally administrable) compositions are known or apparent to those skilled in the art and are described in more detail in, for example, Remington: The Science and Practice of Pharmacy, Pharmaceutical Press; 22nd ed. (2012).

The inventive CAR construct materials may be administered in any suitable manner. Preferably, the inventive CAR construct materials are administered by injection, (e.g., subcutaneously, intravenously, intratumorally, intraarterially, intramuscularly, intradermally, interperitoneally, or intrathecally). Preferably, the inventive CAR construct materials are administered intravenously. A suitable pharmaceutically acceptable carrier for the inventive CAR construct material for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, Ill.), PLASMA-LYTE A (Baxter, Deerfield, Ill.), about 5% dextrose in water, or Ringer's lactate. In an embodiment, the pharmaceutically acceptable carrier is supplemented with human serum albumen.

An “effective amount” or “an amount effective to treat” refers to a dose that is adequate to prevent or treat cancer in an individual. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the active selected, method of administration, timing and frequency of administration, the existence, nature, and extent of any adverse side-effects that might accompany the administration of a particular active, and the desired physiological effect. It will be appreciated by one of skill in the art that various diseases or disorders could require prolonged treatment involving multiple administrations, perhaps using the inventive CAR construct materials in each or various rounds of administration. By way of example and not intending to limit the invention, when the inventive CAR construct material is a host cell, an exemplary dose of host cells may be a minimum of one million cells (1×10⁶ cells/dose).

For purposes of the invention, the amount or dose of the inventive CAR construct material administered should be sufficient to effect a therapeutic or prophylactic response in the subject or animal over a reasonable time frame. For example, the dose of the inventive CAR construct material should be sufficient to bind to antigen, or detect, treat or prevent cancer in a period of from about 2 hours or longer, e.g., about 12 to about 24 or more hours, from the time of administration. In certain embodiments, the time period could be even longer. The dose will be determined by the efficacy of the particular inventive CAR construct material and the condition of the animal (e.g., human), as well as the body weight of the animal (e.g., human) to be treated.

For purposes of the invention, an assay, which comprises, for example, comparing the extent to which target cells are lysed and/or IFN-γ or IL-2 is secreted by T cells expressing the released CARs of the inventive CAR construct upon administration of a given dose of such T cells to a mammal, among a set of mammals of which is each given a different dose of the T cells, could be used to determine a starting dose to be administered to a mammal. The extent to which target cells are lysed and/or IFN-γ or IL-2 is secreted upon administration of a certain dose can be assayed by methods known in the art.

When the inventive CAR construct materials are administered with one or more additional therapeutic agents, one or more additional therapeutic agents can be coadministered to the mammal. By “coadministering” is meant administering one or more additional therapeutic agents and the inventive CAR construct materials sufficiently close in time such that the inventive CAR construct materials can enhance the effect of one or more additional therapeutic agents, or vice versa. In this regard, the inventive CAR construct materials can be administered first and the one or more additional therapeutic agents can be administered second, or vice versa. Alternatively, the inventive CAR construct materials and the one or more additional therapeutic agents can be administered simultaneously. An exemplary therapeutic agent that may be coadministered with the CAR construct materials is IL-2.

It is contemplated that the inventive CAR construct materials can be used in methods of treating or preventing a disease in a mammal. Without being bound to a particular theory or mechanism, the inventive CAR constructs have biological activity, e.g., CARs that recognize antigen, e.g., CD33, such that the CARs, when expressed by a cell, are able to mediate an immune response against the cell expressing the antigen, e.g., CD33. In this regard, an embodiment of the invention provides a method of treating or preventing cancer in a mammal, comprising administering to the mammal any of the CAR constructs, the nucleic acids, the recombinant expression vectors, the host cells, the population of cells, and/or the pharmaceutical compositions of the invention in an amount effective to treat or prevent cancer in the mammal.

An embodiment of the invention further comprises lymphodepleting the mammal prior to administering the inventive CAR construct materials. Examples of lymphodepletion include, but may not be limited to, nonmyeloablative lymphodepleting chemotherapy, myeloablative lymphodepleting chemotherapy, total body irradiation, etc.

For purposes of the inventive methods, wherein host cells or populations of cells are administered, the cells can be cells that are allogeneic or autologous to the mammal. Preferably, the cells are autologous to the mammal.

The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human.

With respect to the inventive methods of treatment, the cancer can be any cancer, including any of acute lymphocytic cancer, acute myeloid leukemia, alveolar rhabdomyosarcoma, bladder cancer (e.g., bladder carcinoma), bone cancer, brain cancer (e.g., medulloblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia (CLL), chronic myeloid cancer, colon cancer, esophageal cancer, cervical cancer, fibrosarcoma, gastrointestinal carcinoid tumor, head and neck cancer (e.g., head and neck squamous cell carcinoma), Hodgkin lymphoma, hypopharynx cancer, kidney cancer, larynx cancer, leukemia, liquid tumors, liver cancer, lung cancer (e.g., non-small cell lung carcinoma), lymphoma, malignant mesothelioma, mastocytoma, melanoma, multiple myeloma, nasopharynx cancer, non-Hodgkin lymphoma, B-chronic lymphocytic leukemia, B-precursor acute lymphoblastic leukemia (B-ALL), pre-B cell precursor acute lymphoblastic leukemia (BCP-ALL), B cell lymphoma, hairy cell leukemia, acute lymphocytic leukemia (ALL), and Burkitt's lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, and ureter cancer. Preferably, the cancer is a hematological malignancy (e.g., leukemia or lymphoma, including but not limited to Hodgkin lymphoma, non-Hodgkin lymphoma, CLL, acute lymphocytic cancer, acute myeloid leukemia, B-chronic lymphocytic leukemia, hairy cell leukemia, acute lymphocytic leukemia (ALL) (also referred to as “acute lymphoblastic leukemia”), B-ALL, BCP-ALL, B cell lymphoma, and Burkitt's lymphoma). Preferably, the cancer is characterized by the expression of CD33.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

Another embodiment of the invention provides a use of the inventive CAR constructs, nucleic acids, recombinant expression vectors, host cells, populations of cells, or pharmaceutical compositions, for the treatment or prevention of cancer in a mammal.

Another embodiment of the invention provides a method of detecting the presence of cancer in a mammal, comprising: (a) contacting a sample comprising one or more cells from the mammal with the CAR constructs, the nucleic acids, the recombinant expression vectors, the host cells, the population of cells, or the pharmaceutical compositions of the invention, thereby forming a complex, (b) and detecting the complex, wherein detection of the complex is indicative of the presence of cancer in the mammal.

The sample may be obtained by any suitable method, e.g., biopsy or necropsy. A biopsy is the removal of tissue and/or cells from an individual. Such removal may be to collect tissue and/or cells from the individual in order to perform experimentation on the removed tissue and/or cells. This experimentation may include experiments to determine if the individual has and/or is suffering from a certain condition or disease-state. The condition or disease may be, e.g., cancer.

With respect to an embodiment of the inventive method of detecting the presence of cancer in a mammal, the sample comprising cells of the mammal can be a sample comprising whole cells, lysates thereof, or a fraction of the whole cell lysates, e.g., a nuclear or cytoplasmic fraction, a whole protein fraction, or a nucleic acid fraction. If the sample comprises whole cells, the cells can be any cells of the mammal, e.g., the cells of any organ or tissue, including blood cells or endothelial cells.

For purposes of the inventive detecting method, the contacting can take place in vitro or in vivo with respect to the mammal. Preferably, the contacting is in vitro.

Also, detection of the complex can occur through any number of ways known in the art. For instance, the inventive CAR constructs, nucleic acids, recombinant expression vectors, host cells, or populations of cells, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

Methods of testing a CAR for the ability to recognize target cells and for antigen specificity are known in the art. For instance, Clay et al., J Immunol., 163: 507-513 (1999), teaches methods of measuring the release of cytokines (e.g., interferon-γ, granulocyte/monocyte colony stimulating factor (GM-CSF), tumor necrosis factor a (TNF-α) or interleukin 2 (IL-2)). In addition, CAR function can be evaluated by measurement of cellular cytoxicity, as described in Zhao et al., J. Immunol., 174: 4415-4423 (2005).

The following includes certain aspects of the invention.

1. A chimeric antigen receptor (CAR) comprising an antigen binding domain having antigenic specificity for CD33, a transmembrane domain, and an intracellular T cell signaling domain, wherein

(a) the antigen binding domain comprises the light chain variable region comprising the CDR1, CDR2, and CDR3 regions of Hu195; or

(b) the antigen binding domain comprises the heavy chain variable region comprising the CDR1, CDR2, and CDR3 regions of Hu195, wherein the CDR regions are those of SEQ ID NOS: 41-46.

2. The CAR according to aspect 1, wherein the antigen binding domain comprises the heavy chain variable region of SEQ ID NO: 15.

3. The CAR according to aspect 1 or 2, wherein the antigen binding domain comprises the light chain variable region of SEQ ID NO: 16.

4. The CAR according to any one of aspects 1-3, wherein the antigen binding domain comprises a linker sequence of SEQ ID NO: 4.

5. The CAR according to any one of aspects 1-4, wherein the antigen binding domain comprises the antigen binding domain of SEQ ID NOS: 15, 4, and 16.

6. The CAR according to any one of aspects 1-5, wherein the CAR comprises (i) a CD8 transmembrane domain of SEQ ID NO: 7 and a CD8 hinge domain of SEQ ID NO: 6, or (ii) a CD28 transmembrane domain of SEQ ID NO: 11 and a CD28 hinge domain of SEQ ID NO: 10.

7. The CAR according to any one of aspects 1-6, wherein the intracellular T cell signaling domain comprises the 4-1BB intracellular T cell signaling domain of SEQ ID NO: 8, the CD3 zeta intracellular T cell signaling domain of SEQ ID NO: 9, or both.

8. The CAR according to any one of aspects 1-7, wherein the CAR further comprises a spacer.

9. A chimeric antigen receptor (CAR) comprising an antigen binding domain having antigenic specificity for CD33, a transmembrane domain, and an intracellular T cell signaling domain, wherein

(a) the antigen binding domain comprises the light chain variable region comprising the CDR1, CDR2, and CDR3 regions of hP67.6; and/or

(b) the antigen binding domain comprises the heavy chain variable region comprising the CDR1, CDR2, and CDR3 regions of hP67.6

wherein the CDR regions are those of SEQ ID NOS: 47-52.

10. The CAR according to aspect 9, wherein the antigen binding domain comprises the heavy chain variable region of SEQ ID NO: 3.

11. The CAR according to aspect 9 or 10, wherein the antigen binding domain comprises the light chain variable region of SEQ ID NO: 5.

12. The CAR according to any one of aspects 9-11, wherein the antigen binding domain comprises a linker sequence of SEQ ID NO: 4.

13. The CAR according to any one of aspects 9-12, wherein the antigen binding domain comprises the antigen binding domain of SEQ ID NOS: 3, 4, and 5.

14. The CAR according to any one of aspects 9-13, wherein the CAR comprises (i) a CD8 transmembrane domain of SEQ ID NO: 7 and a CD8 hinge domain of SEQ ID NO: 6, or (ii) a CD28 transmembrane domain of SEQ ID NO: 11 and a CD28 hinge domain of SEQ ID NO: 10.

15. The CAR according to any one of aspects 9-14, wherein the intracellular T cell signaling domain comprises the 4-1BB intracellular T cell signaling domain of SEQ ID NO: 8, the CD3 zeta intracellular T cell signaling domain of SEQ ID NO: 9, or both.

16. The CAR according to any one of aspects 9-15, wherein the CAR further comprises a spacer.

17. A CAR comprising SEQ ID NO: 16 or 17 or 20 or 21.

18. A nucleic acid comprising a nucleotide sequence encoding the CAR according to any one of aspects 1-17.

19. The nucleic acid according to aspect 18, wherein the nucleotide sequence is codon-optimized.

20. A recombinant expression vector comprising the nucleic acid according to aspect 18 or 19.

21. An isolated host cell comprising the recombinant expression vector of aspect 20.

22. A population of cells comprising at least one host cell of aspect 21.

23. A pharmaceutical composition comprising the CAR any one of aspects 1-17, the nucleic acid of aspect 18 or 19, the recombinant expression vector of aspect 20, the host cell of aspect 21, or the population of cells of aspect 22, and a pharmaceutically acceptable carrier.

24. A method of detecting the presence of cancer, comprising:

(a) contacting a sample comprising one or more cells with the CAR any one of aspects 1-17, the nucleic acid of aspect 18 or 19, the recombinant expression vector of aspect 20, the host cell of aspect 21, or the population of cells of aspect 22, or the pharmaceutical composition of aspect 23, thereby forming a complex, and

(b) detecting the complex, wherein detection of the complex is indicative of the presence of cancer.

25. The method of aspect 24, wherein the cancer is acute myeloid leukemia.

26. The CAR of any one of aspects 1-17, the nucleic acid of aspect 18 or 19, the recombinant expression vector of aspect 20, the host cell of aspect 21, the population of cells of aspect 22, or the pharmaceutical composition of aspect 23, for use in the treatment or prevention of cancer in a mammal.

27. The CAR, nucleic acid, recombinant expression vector, host cell, population of cells, or pharmaceutical composition of aspect 26, wherein the cancer is acute myeloid leukemia.

28. A method of treating or preventing cancer in a mammal, the method comprising administering to the mammal an effective amount of the CAR of any one of aspects 1-17, the nucleic acid of aspect 18 or 19, the recombinant expression vector of aspect 20, the host cell of aspect 21, the population of cells of aspect 22, or the pharmaceutical composition of aspect 23.

29. The method of aspect 29, wherein the cancer is acute myeloid leukemia.

The following examples further illustrate the invention but, of course, should not be construed as in any way limiting its scope.

### Example 1

This example demonstrates the use of CARs in accordance with embodiments of the invention.

### CAR Constructs

CAR constructs were developed with target specific single chain fragment variable sequences (scFv), linked with transmembrane domains, and paired with either 4-1BB or CD28 co-stimulatory domains, CD3 zeta signaling domains, and cloned in the third-generation lentiviral plasmid. The scFv of CD33 CAR constructs were derived from the following: Lintuzumab (Hu195, SGN-33) (Co et al., J. Immunol., 148: 1149-54 (1992)), and CD33Mylo (gemtuzumab ozogamicin, Trade name: Mylotarg, Company: Wyeth, humanized mAb/calicheamicin, CD33; U.S. Pat. No. 5,739,116; Cowan et al., Front Biosci (Landmark Ed)., 18: 1311-34 (2013)). The CD123 CAR was derived from 32716-scFv (International Patent Application Publication No. WO 2014/144622). These CARs were subcloned into an pELNS lenti vector backbone. All restriction enzymes were purchased from New England Biolabs (Ipswich, Mass., USA). The sequences of all CAR constructs was confirmed by sequencing at Macrogen (Rockville, Md., USA). See FIGS. 1A and 1B.

### Cell Lines

The GFP and luciferase expressing AML cells lines MV411, THP1, and MOLM14 contain varying levels of CD33 expression, and different genotypes for an exon 2 splice variance (Laszlo et al., Oncotarget, 7: 43281-94 (2016)) were used to test CAR efficacy. Through DNA isolation, it was found that MOLM14 has a CC genotype and does not contain the SNP, while THP1 and MV411 are both heterozygous for the SNP with the CT genotype (Lamba et al., J. Clin. Oncol., 35: 2674-82 (2017)). This cell line does not express neither CD33 nor CD123. MV411 is an acute monocytic leukemia line established from a 10-year-old boy with acute monocytic leukemia (AML FAB M5). MOLM14 is an acute myeloid leukemia line established from the peripheral blood of a 20-year-old man with acute myeloid leukemia AML FAB M5a at relapse in 1995 after initial myelodysplastic syndrome (MDS, refractory anemia with excess of blasts, RAEB). THP-1 is a human monocytic cell line derived from an acute monocytic leukemia patient. K562 is a human erythroleukemia leukemia line established and derived from a 53-year-old female chronic myelogenous leukemia patient.

### CAR T-Cell Generation

The CD33 or CD123 CAR-encoding lentiviral vectors were produced by transient transfection of the Lenti-X 293T lenti packaging cell line. Lenti-X 293T cells were plated into poly-D lysine coated 15-cm plates (BD Biosciences, San Jose, Calif., USA). The following day, Lenti-X 293T cells were transfected using lipofectamine 3000 (Thermo Fisher Scientific, Waltham, Mass., USA) with plasmids encoding the CAR along with packaging and envelope vectors (pMDLg/pRRE, pMD-2G, and pRSV-Rev). Lentiviral supernatants were harvested at 24 and 48 hours post-transfection, centrifuged at 3000 RPM for 10 minutes to remove cell debris, frozen on dry ice and stored at −80° C. Human PBMCs from normal donors were obtained with an NIH-approved protocol and activated with a 1:3 ratio of CD3/CD28 microbeads (Dynabeads Human T-Expander CD3/CD28, Thermo Fisher Scientific, Cat #11141D) in AIM-V media containing 40 IU/mL recombinant IL-2 and 5% FBS for 24 hours. Activated T cells were resuspended at 2 million cells per 2 mL of lentiviral supernatant plus 1 mL of fresh AIM-V media with 10 mcg/mL protamine sulfate and 100 IU/mL IL-2 in 6-well plates. Plates were centrifuged at 1000×g for 2 hours at 32° C. and incubated overnight at 37° C. A second transduction was performed on the following day by repeating the same transduction procedure described above. The CD3/CD28 beads were removed on the third day following transduction, and the cells were cultured at 300,000 cells/mL in AIM-V containing 100 IU/mL IL2 with fresh IL2-containing media added every 2-3 days until harvest on day 8 or 9.

### Flow Cytometry

Surface expression of CD33 CAR-transduced T cells was determined by flow cytometry using either protein-L (Themo Fisher) or a Biotinylated Human Siglec-3/CD33 Protein (Acro Biosystems, Newark, Del., USA) followed by incubation with Streptavidin-PE (BioLegend, San Diego, Calif., USA). CD123 CAR expression were detected with protein-L. Expression of CD33, CD123 and other cell surface markers were detected using the following antibodies from eBioscience (Thermo Fisher): CD33, CD45, CD3, CD8a, CD4, CD10.

### PDX

1 million of PDX leukemia cell JMM117 were injected into the NSG mice one week ahead of adoptive CAR T cell transfer. The mice were treated with CAR T cells on day 0. Two weeks later the mice were taken down and analysis was performed for the leukemia cell and the CAR T cells.

### Cytotoxicity Assay

5E4 of Target tumor cells in 100 ul of RPMI media were loaded into a 96-well plate (Corning® (Corning, N.Y.) BioCoat™ Poly-L-Lysine 96-Well Clear TC-Treated Flat Bottom Assay Plate). An equal amount of CAR T cells were added into the designated well on the following day. The initial incucyte apoptosis marker (Essen BioScience, Ann Arbor, Mich., USA) was diluted in 100 ul PBS and 1 ul of the diluent was added into each well. The plate was scanned for the GFP and or RFP fluorescent expression to monitor the cell apoptosis using an IncuCyte ZOOM® system every 30 minutes in a duration of 40 hours. The percentage of cell killing at each time point was baseline-corrected.

### Analysis of Cytokine Production

Target tumor cell and transduced CAR positive T cells were washed 3 times with 1×PBS and resuspended in RPMI at 1E6/ml. 100 ul of tumor cells with 100 ul of CAR positive T cells were loaded into each well of a 96-well plate. T cell only and tumor cell only controls were set up. All tests were performed in duplicate or triplicate. Cells were incubated for 18 hours at 37° C. and 120 ul of the culture supernatant was harvested for detection of cytokine production. Cytokine levels in supernatants were measured using either ELISA kits (R&D Systems, Minneapolis, Minn., USA) or a multiplex assay (Meso Scale Discovery, Rockville, Md., USA).

### Bioenergetic Analyses

For the glycolysis stress test, the CAR-T cells were suspended in serum-free unbuffered DMEM medium (Sigma-Aldrich, St. Louis, Mo., USA) supplemented with L-glutamine (200 mM) and NaCl (143 mM). 0.6 mL of a 0.5% Phenol Red solution (SigmaP0290) was added for a final concentration of 3 mg/L and adjust the pH to 7.35+/−0.05. CAR-T cells were plated onto Seahorse cell plates (3E5 cells per well), coated with Cell-Tak (Corning) to facilitate T cell attachment. Briefly, the cartridges were hydrated the day before the assay. On the day of the assay, the plates were coated with Cell-Tak and the cells were seeded in the Cell-Tak coated plates and placed on the XF24 Analyzer for the assay. The detailed procedure is as follows. The assay cartridge was initially hydrated with XF calibrant solution at 200 ul/well, hydro booster was added, and wrapped in parafilm, and the sensor cartridge was placed on top of utility plate and incubated at 37° C. without C0₂ for overnight. The cell culture plate was then coated with Cell-Tak as follows: For 1 plate, 46 μl of Cell-Tak was diluted in 2040 TC water and 1 ml of NaHCO₃. The mixer was dispensed 50 μl in each well and the plate was incubated at room temperature for at least 20 minutes. After removing the Cell-Tak solution, 250 μl of TC water was used to wash each well. CAR-T cells (3E5/well) were plated in 158 μl assay media. The cell culture plate was then spun at 450 rpm for 1 sec at slow acceleration and no deceleration, and then the plate was reversed in orientation and spun at 650 rpm for 1 sec at slow acceleration and no deceleration. The plate was then incubated at 37° C. 0% CO₂ for 25-30 minutes. After 25-30 minutes incubation, 158 ul of warm assay medium was added slowly and gently to the top of each well along the side of the wall using a manual P200 pipettor. The cell plates were incubated for 15-25 minutes. After 15-25 minutes, the plates were placed on XF24 Analyzer (after calibration finished). The XF assay was executed. Solution was injected sequentially through three ports: Port A: glucose 80 mM (96 μl of the stock solution in 3 ml assay media). Port B: oligomycin 18 μM (10.8 μl of the stock solution in 3 ml assay media). Port C: 2DG use stock solution. Glycolysis stress test was performed by measuring ECAR (mpH/min) at steady state after the cartridge ports were loaded with 75 μl of drug solution. For the mitochondrial stress test, CAR T cells were suspended in serum-free unbuffered DMEM medium with D-glucose (25 mM), and sodium pyruvate (1 mM). Mitochondrial stress test was performed similarly as the above by measuring OCR (pmol/min) at steady state and after sequential injection of oligomycin (0.5 μM), FCCP (0.5 μM), rotenone (1 μM) and antimycin A (1 μM) (Sigma-Aldrich). Experiments with the Seahorse system utilized the following assay conditions: 2 minutes mixture; 2 minutes wait; and 3 minutes measurement. All samples were tested in six replicates.

### Fluorescence Microscopy Imaging and Analysis

MOLM14 (4×10⁵) tumor cells were plated in 1 ml of warm RPMI on the Cell-tak coated inner well of an ibidi μ-Dish 35 mm and incubated overnight in a 37 C incubator. Tumor cells were then stained with Hoechst Dye (2.5 ug/ml). T cells were transduced to express CAR-mCherry fusion proteins. CAR-T positive cells were sorted and then 7.5 E5 of these CAR-T cells were incubated with the fixed MOLM14 cell in the dish for an hour. The cells were subsequently washed and fixed with freshly prepared 4% paraformaldehyde and mounted in a non-hardening mounting media in preparation for imaging.

To evaluate actin expression at the immune synapse, the above protocol was modified, and samples were permeabilized with 0.1% triton x after paraformaldehyde fixation. Cells were stained with Phalloidin 640 (165 nM) and then washed prior to mounting.

Airyscan images were acquired using a Zeiss LSM 880. The exposure setting was the same for the entire experiment. Images were collected as a z stack to cover the entire volume of the immune synapse.

Some images were acquired using a Nikon Eclipse Ti2 spinning disc confocal microscope with 63× objective. Z stacks of 0.5 uM thickness were acquired in parallel over a range of 10 uM above and below the focal plane for the three channels (405, 488, 640 nm). Each channel was excited at 50% laser intensity with exposure times of 300 ms, 1 s, and 300 ms for 405, 488, and 640, respectively. ImageJ software was used for data analysis.

CAR aggregation at the immune synapse (IS) was observed for both CD33-28 and CD33-BBz CAR constructs. Increased accumulation of F-actin was correlatively observed at the IS relative to unengaged cells for both CAR constructs. Quantitative analysis for n>10 immune synapses for each CAR were performed to evaluate CAR and actin accumulation. Specifically, the ratio of mean fluorescence intensity (MFI) at the synapse vs. ratio of the MFI at the rest of the T cell surface were determined. Additional parameters include ratio of MFI*volume at the IS vs. MFI*volume for the rest of the T cell surface, MFI*volume of IS vs. MFI*volume of T cell, and intracellular CAR signal vs. extracellular CAR signal were also evaluated. For actin, fluorescence intensity at the IS were normalized against the baseline actin T cell expression. MFI*volume of actin at the IS were determined and MFI*volume of unengaged T and tumor cells were subtracted to account for baseline actin expression.

The sequence for mCherry is:

(SEQ ID NO: 40)
ATGGTGAGCAAGGGCGAGGAGGATAACATGGCCATCATCAAGGAGTTCAT
GCGCTTCAAGGTGCACATGGAGGGCTCCGTGAACGGCCACGAGTTCGAGA
TCGAGGGCGAGGGCGAGGGCCGCCCCTACGAGGGCACCCAGACCGCCAAG
CTGAAGGTGACCAAGGGTGGCCCCCTGCCCTTCGCCTGGGACATCCTGTC
CCCTCAGTTCATGTACGGCTCCAAGGCCTACGTGAAGCACCCCGCCGACA
TCCCCGACTACTTGAAGCTGTCCTTCCCCGAGGGCTTCAAGTGGGAGCGC
GTGATGAACTTCGAGGACGGCGGCGTGGTGACCGTGACCCAGGACTCCTC
CCTGCAGGACGGCGAGTTCATCTACAAGGTGAAGCTGCGCGGCACCAACT
TCCCCTCCGACGGCCCCGTAATGCAGAAGAAGACCATGGGCTGGGAGGCC
TCCTCCGAGCGGATGTACCCCGAGGACGGCGCCCTGAAGGGCGAGATCAA
GCAGAGGCTGAAGCTGAAGGACGGCGGCCACTACGACGCTGAGGTCAAGA
CCACCTACAAGGCCAAGAAGCCCGTGCAGCTGCCCGGCGCCTACAACGTC
AACATCAAGTTGGACATCACCTCCCACAACGAGGACTACACCATCGTGGA
ACAGTACGAACGCGCCGAGGGCCGCCACTCCACCGGCGGCATGGACGAGC
TGTACAAG

### In Vivo Experiments

Animal experiments were carried out under protocols approved by the NCI Bethesda Animal Care and Use Committee. AML cell lines and the xenografted human AML specimens were IV injected into NSG mice. For luciferase-expressing lines, leukemia was detected using the Xenogen IVIS Lumina (Caliper Life Sciences, Hopkinton, Mass., USA). NSG were injected intraperitoneally with 3 mg D-luciferin (Caliper Life Sciences) and were imaged 4 minutes later with an exposure time of 1 min for AML cell lines. Living Image Version 4.1 software (Caliper Life Sciences) was used to analyze the total bioluminescent signal flux for each mouse as photons/s. At time of take down, bone marrow, spleen, and liver of mice were harvested assessed by flow cytometry.

### Statistical Analysis

Statistics analysis were performed using Prism 7.0 software. Plots are presented as mean+/−SD. Statistical significance of all data was calculated using an unpaired student t test. p<0.05 was considered as significant.

### Development of CD33 and CD123 CARs

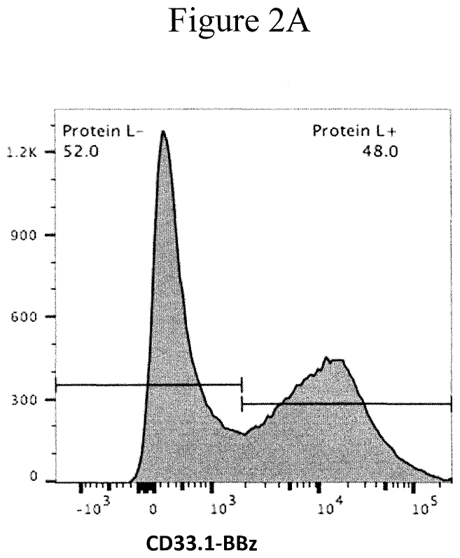

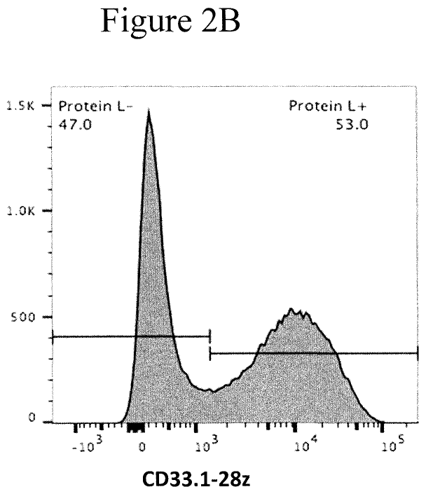

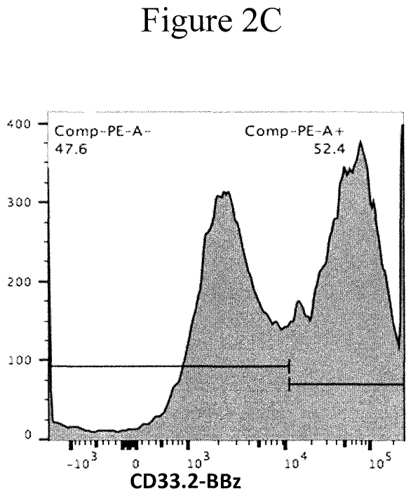

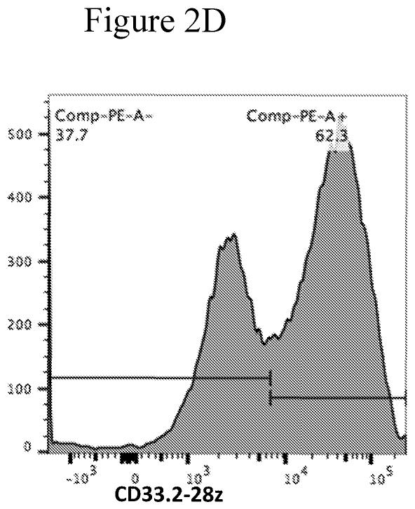

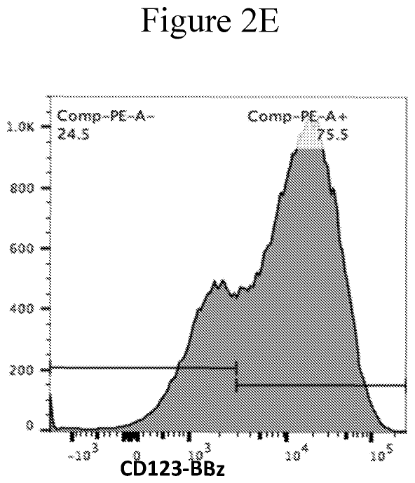

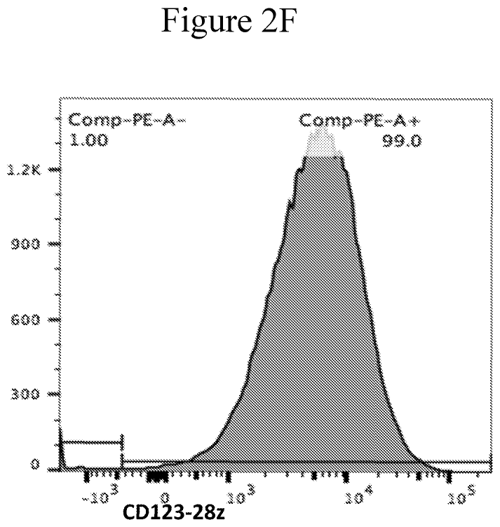

Second-generation CARs were developed using two scFv combined with either the 4-1BB or CD28 co-stimulatory domain. For the anti-CD33 CARs, CD33.1 CAR contains the gemtuzumab antibody, which is also known as Mylotarg; CD33.2 CAR contains the antibody known as lintuzumab, or the humanized M195 (Hu195). The anti-CD123 CAR was derived from 32716, a mouse monoclonal antibody that specifically binds to human CD123. Post transduction, protein L detection showed that CARs with the same scFv have a similar amount of transduction efficiency, irrespective of the co-stimulatory domain. Differences in transduction efficiency appear to be related to the scFv. In CD123 CAR, the transduction efficiency is higher; however, the CAR surface expression density looks lower than the CD33 CARs in general. See FIGS. 2A-2F.

### Evaluating CD33 and CD123 Surface Expression on AML

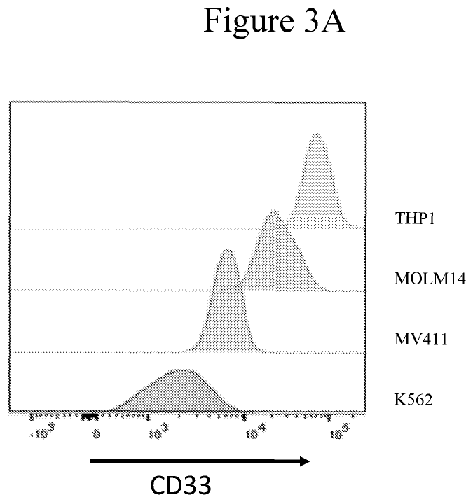

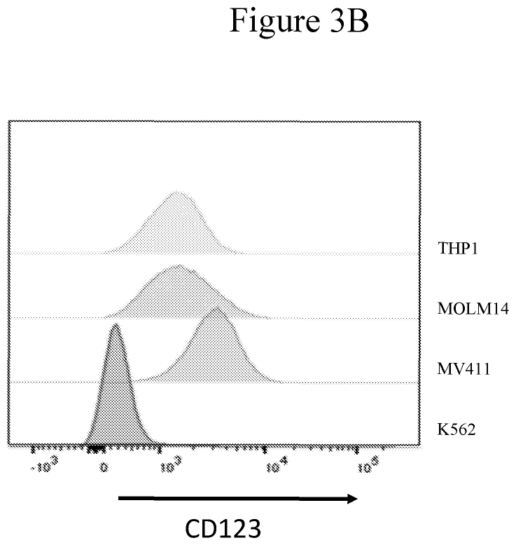

Four cells lines, K562, MV411, MOLM14, and THP1, were evaluated using anti-CD33 flow antibodies for the surface expression of CD33. MV411, MOLM14, and THP1 express CD33 in ascending order of increased surface expression of CD33. For CD123 surface expression, there is increased surface expression in the order of K562, THP1, MOLM14, and MV411. These three AML cell lines present a wide range of surface expression and thus were chosen for further experiments. See FIGS. 3A and 3B.

### In Vitro Cytokine Production and Cytotoxicity Assays Confirm CAR Activity Against Tumor Targets

In vitro cytokine assays were used to evaluate the efficacy of the AML CARs. In general, the cytokine production correlated with the level of target antigen expression and revealed that the CD28 equipped CARs consistently produced more interferon-gamma than 4-1BB equipped CARs across multiple AML cell lines. CD33.2 and CD123 CARs made less IFN-gamma than CD33.1 CARs in general. Notably, CD33.1 BBz and CD28z, and CD33.2 BBz CARs had some activity with production of IFN-gamma without tumor antigen stimulation while CD33.28z CAR had no detectable level of IFN-gamma.

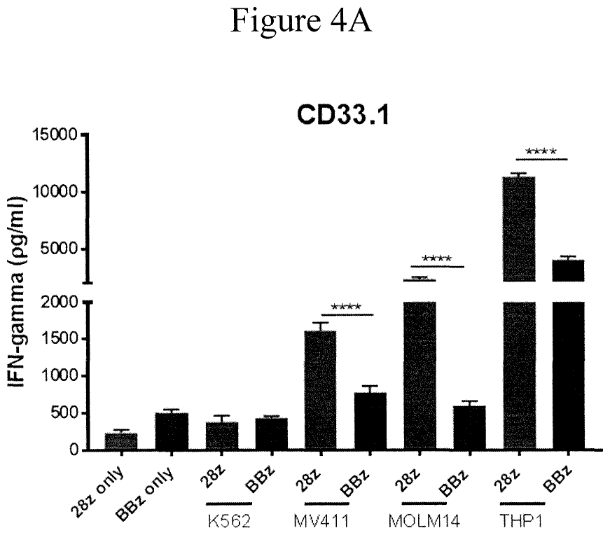

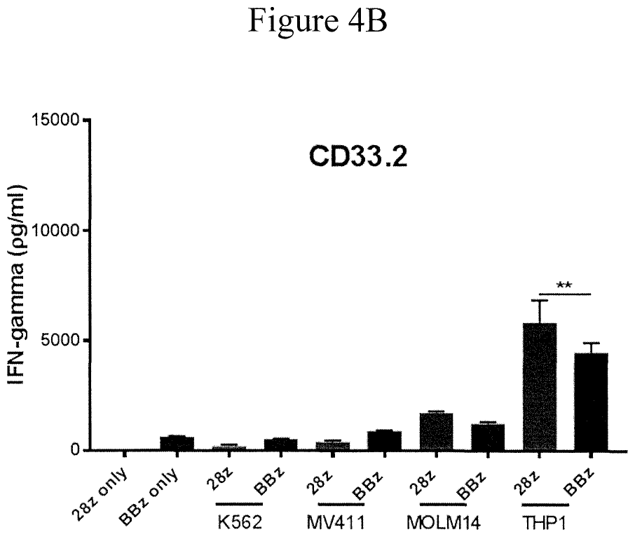

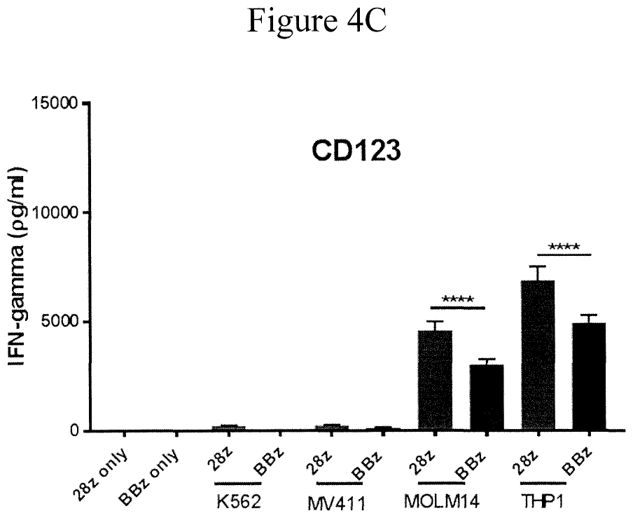

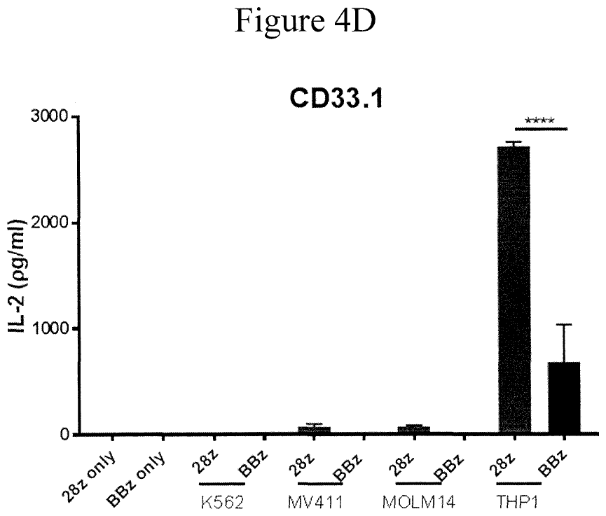

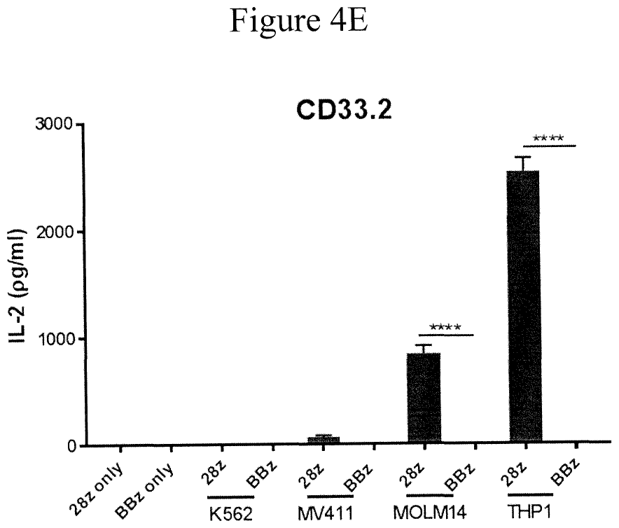

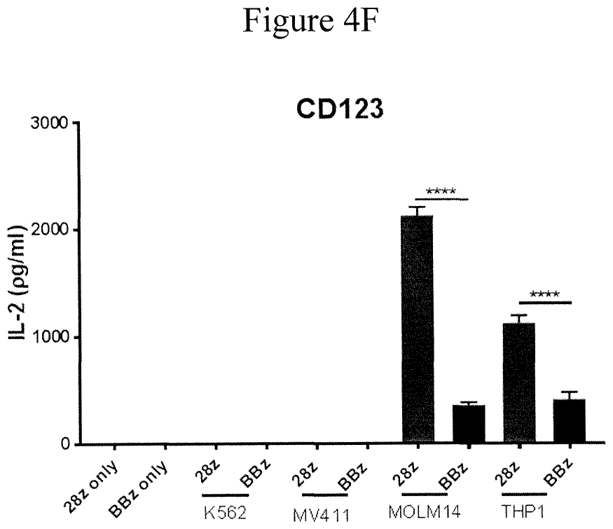

IL-2 has been considered as a more reliable maker for CAR efficacy. CD33.1-28z and CD33.1-BBz produce high amount of IL-2 only when incubated with the CD33 high antigen expression THP1 cell line. In contrast, CD33.2-28z CAR made comparable level of IL-2 THP1 line and a decent amount of IL-2 against MOLM14 which has moderate level of expression of target antigen. CD123 CARs also made a decent amount of IL-2 against MOLM14 and THP1 but no detectable level of IL-2 with MV411. Both CD33.1 and CD33.2 CARs produced low level of IL-2 against MV411 indicating low activity in vivo may due to the low surface antigen expression, also suggesting that there may be low activity in vivo. See FIGS. 4A-4F.

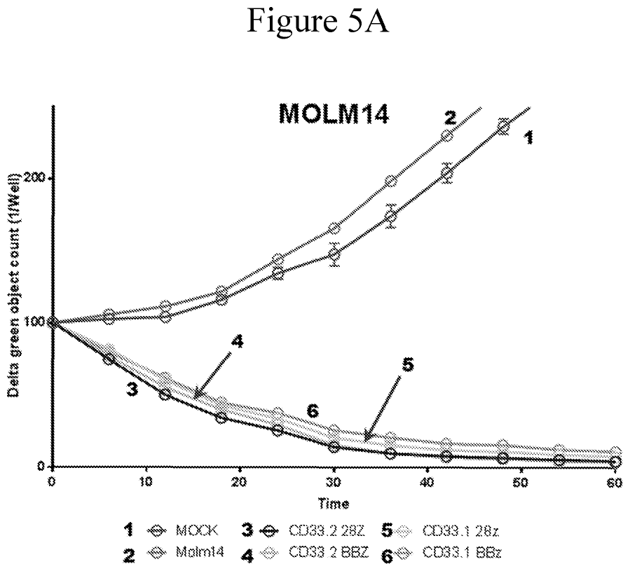

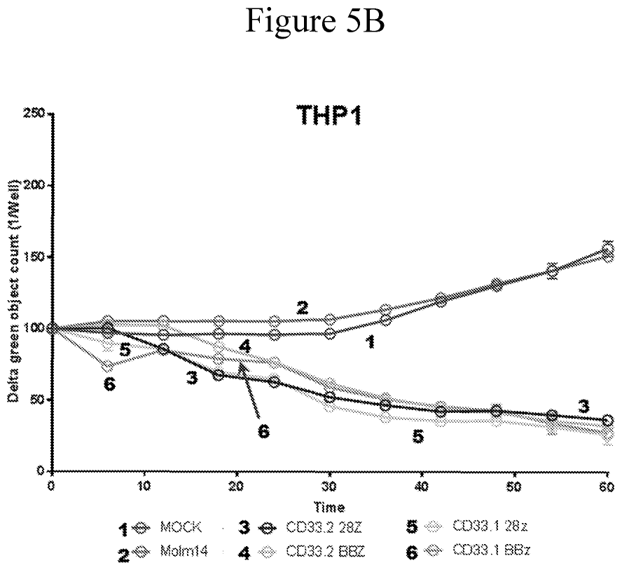

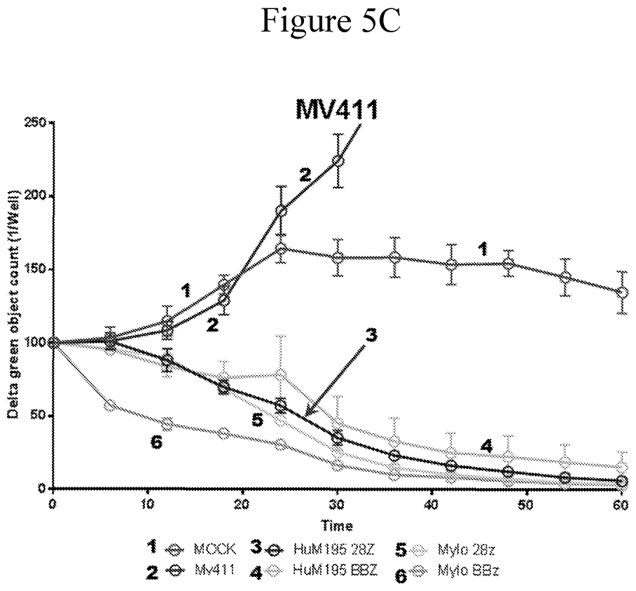

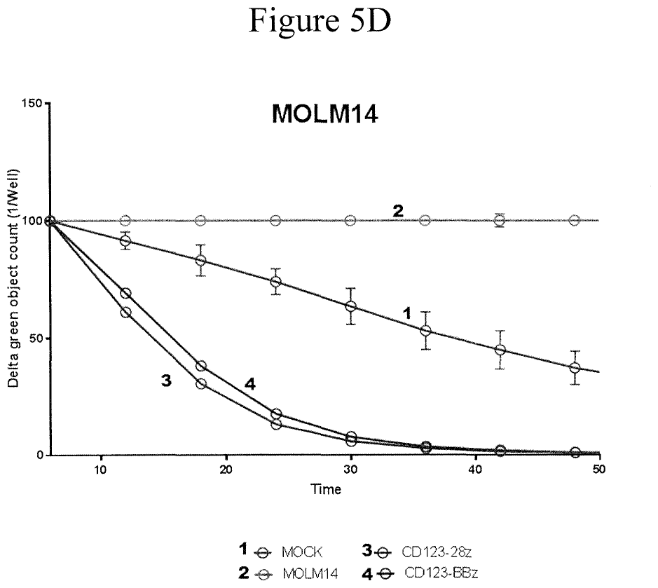

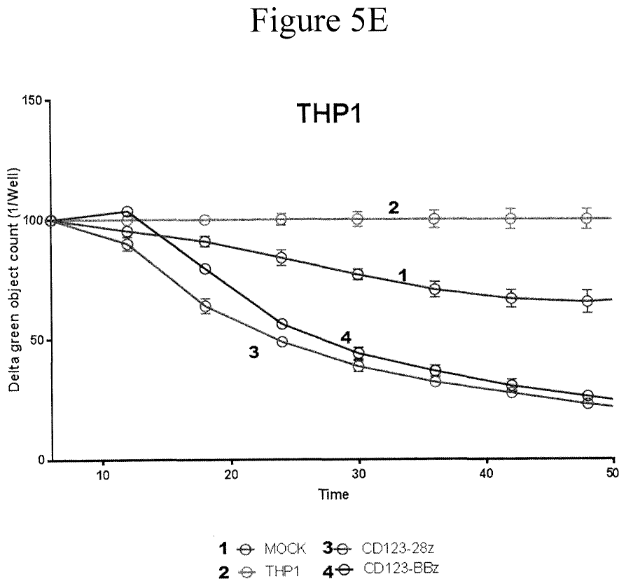

In IncuCyte killing assay, CD33 CAR transduced T cells were incubated with target leukemia cells. The percentage of the live leukemia cell relative to the original plated cell were plotted. The plots demonstrated efficient killing of THP1, MV411, and MOLM14 leukemia in vitro. See FIGS. 5A-5E.

28z CAR is more effective than 4-1BB CAR on AML model and BBz CARs show extramedullary disease relapse patterns

To translate these findings in vivo, xenograft models were injected with THP1 AML cells and treated with either CD33 CAR T cells. By bioluminescence imaging, CD33.1 CARs showed more toxicity than the CD33.2 CARs as seen with decrease in weight, hypothermia, and lethargy. CD33.2-CD28z treated mice had no detectable disease while CD33-4-1 BB treated mice had leukemia which indicate that the 28z equipped CAR is more effective than 4-1BB CAR on eradicating AML. This is different from the observation in ALL model. This phenomenon was further confirmed with CD123 CAR on THP1 model, CD33.1 CAR on MOLM14 model.

Combined, the in vitro and in vivo results suggest the co-stimulatory domain does play a critical role in CAR T cell functionality and may have different impacts in different tumor models. To confirm the presence of AML in mice detected by bioluminescence, flow cytometry was performed on mice tissues. CD33-4-1BB treated animals were clear of any leukemia in the bone marrow, suggesting the presence of extra medullary disease (EMD). The development of EMD in the less potent CD33-4-1BB CAR treated mice suggests that CAR immune pressure may be potent enough to clear primary sites of leukemia such as the bone marrow, but unable to eliminate disease in secondary tissues that AML can seed.

To further investigate the effects of these two co-stimulation factors, another AML model was used, MV411, that regularly presents with EMD even in the absence of CAR pressure. With CD33.2-CD28 against MV411, there was clearance in bone marrow, however CD33.2-CD28 CAR was not able to prevent the development of EMD. These experiments suggest that although the CD28 costimulatory domain is more potent than 4-1BB in THP1, the potency of CD28 is still not able to overcome EMD in all models.

In the MOLM14 model, when tissues were harvested to look at phenotype of the leukemia and CAR T cells in the mice, flow cytometry was used to analysis these cell types. In GFP CAR treated mice, CD33⁺ leukemia and transduced T cells were found in the bone marrow and spleen compartments. In the CD33.1 BBz CAR treated condition, there were low amounts of CD33⁺ leukemia found in the bone marrow and spleen, and large amounts in solid tumors found on the leg and wrapping around the intestines. Flow analysis of the tumor cells show that the AML still retain CD33 surface expression with a shift in a decreased amount of CD33 expression.

To confirm the efficacy of the CD33.2 CAR, a dose titration was performed on the aggressive MOLM14 tumor model. CD33.2-28z CAR can efficiently clear the leukemia with as low as 5 million of CAR′ T cells.

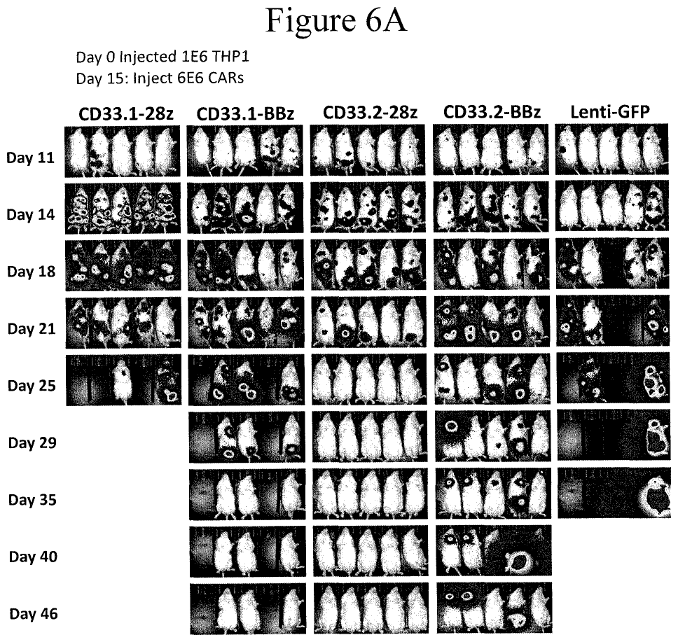

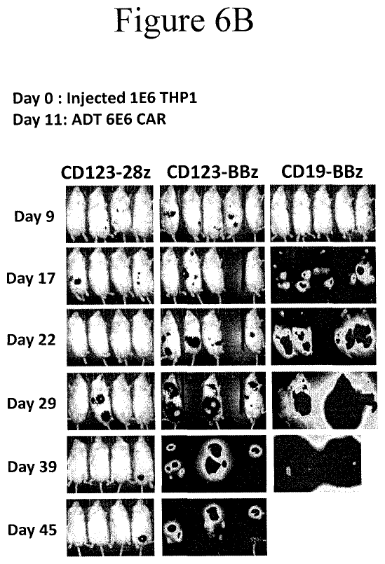

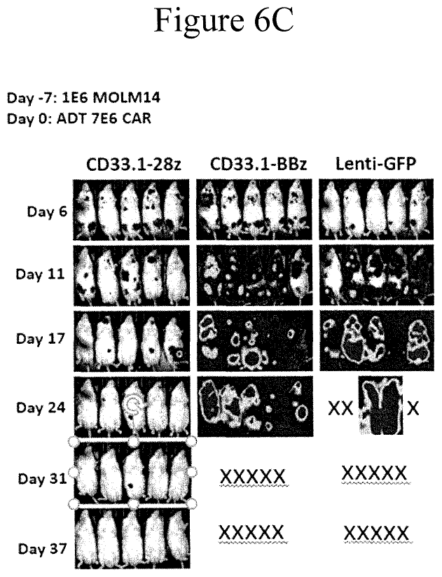

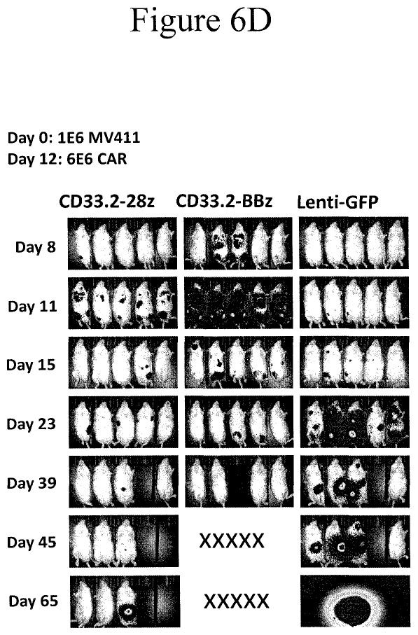

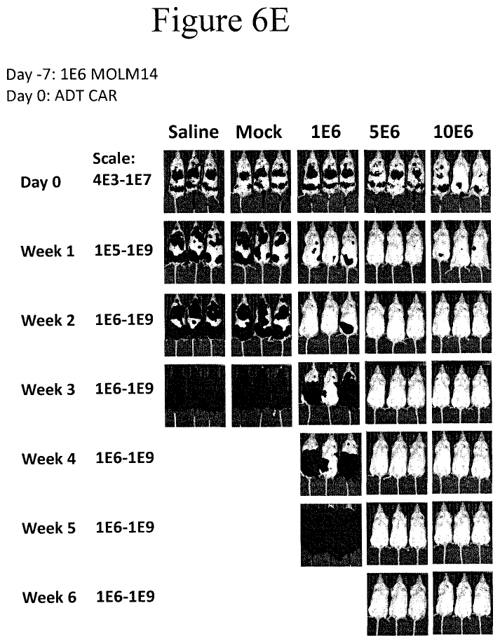

See FIGS. 6A-6E.

### Potent Activity of CD33.2-CD28z CARs on Eradicating PDX

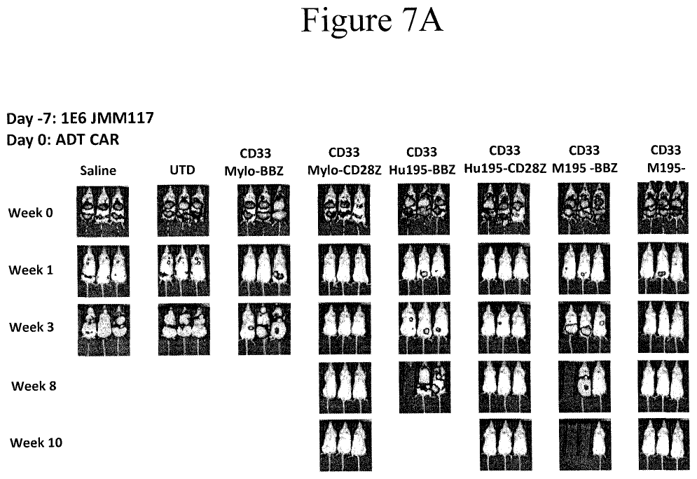

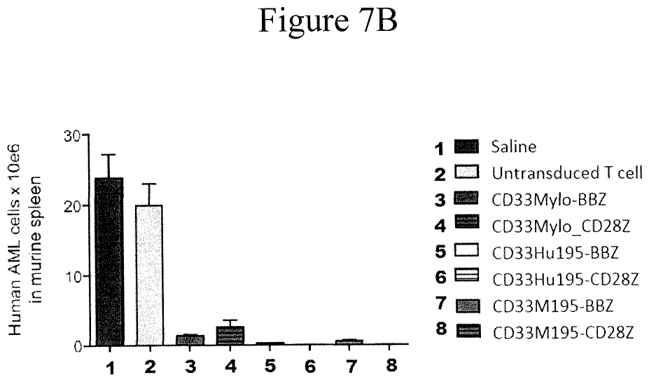

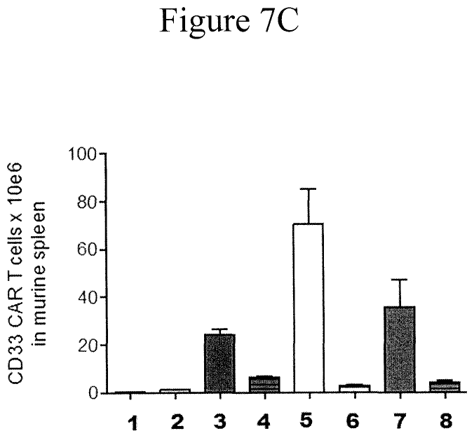

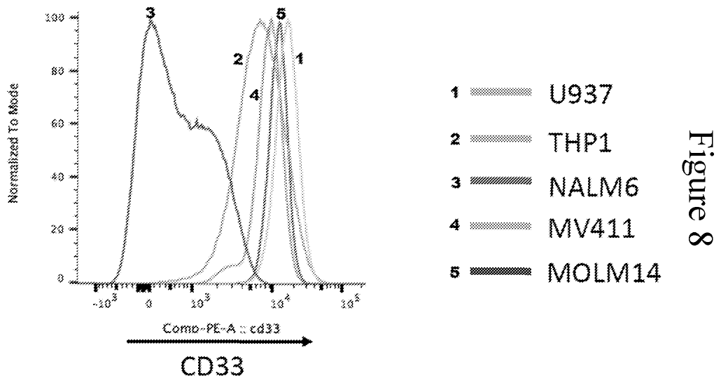

A clinic relevant primary childhood AML PDX model was used to verify the activity of CD33 CARs. One million of PDX Leukemia cell JMM117 were injected on day −7 into NSG mice followed with ADT of 1E6 of CAR T cells on day 0. CD28 CARs were better than BBz CARs, and CD33.2-BBz performs better than the CD33.1-BBz CAR in vivo (FIG. 7A). This was confirmed with flow analysis on week 2 that the CD33.1 treated mouse spleen had detectable levels of leukemia while close to none in CD33.2 CAR groups (FIG. 7B). Furthermore, this PDX model showed that CARs with 41-BBz co-stimulatory domain remain elevated for longer (FIG. 7C). When looking at CAR persistence, it was observed that across both CD33.1 and CD33.2 CARs, the 4-1BBz versions remain detected at much higher amounts than CD28z CAR which may be related with the high toxicity of the BBz CARs in AML model.

The CD28z containing CARs show increased potency compared to 4-1BB CARs, but without increased toxicity. Although prolonged B-cell aplasia may be an acceptable outcome following CD19 CAR-T cell therapy, given the concern for prolonged myelosuppression and persistent aplasia following a myeloid directed CAR-T cell approach, the less persistent CD28z based CAR may be further advantageous by not only more efficiently eradicating disease but then allowing for normal hematopoietic recovery with self-limited persistence of the CAR. Strategies to deplete CD123 CAR, or consideration of hematopoietic stem cell transplantation (HSCT) following an AML directed CAR, represent other strategies to effectively eliminate a persistent CAR and restore effective hematopoiesis.

Based on the above, CD33.2-CD28z CAR is the most potent CAR with less toxicity compared to all the other constructs. The impact of the co-stimulation domain on anti-CD33 CARs is opposite from the observation with respect to anti-CD19 or anti-CD22 CARs.

### Example 2

This example demonstrates flow cytometric analysis of CD33 target antigen expression on Leukemia cells.

See FIG. 8: U937 is a histiocytic lymphoma line of the myeloid lineage isolated from the histiocytic lymphoma of a 37-year-old male patient. THP1 is a human acute monocytic leukemia line cultured from the blood of a 1 year boy with acute monocytic leukemia. NALM6 is a human B cell precursor leukemia line established from the peripheral blood of a 19-year-old man with acute lymphoblastic leukemia. MV411 is an acute monocytic leukemia line established from a 10-year-old boy with acute monocytic leukemia (AML FAB M5). MOLM14 is an acute myeloid leukemia line established from the peripheral blood of a 20-year-old man with acute myeloid leukemia, AML FAB M5a at relapse in 1995 after initial myelodysplastic syndrome (MDS, refractory anemia with excess of blasts, RAEB); carries internal tandem duplication of FLT3; cell line carries the CBL deltaExon8 mutant.

### Example 3

This example demonstrates the use of CARs in accordance with embodiments of the invention.

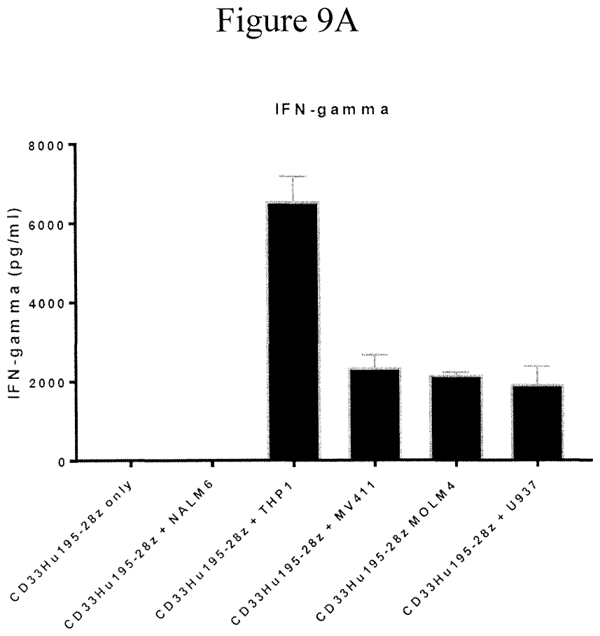

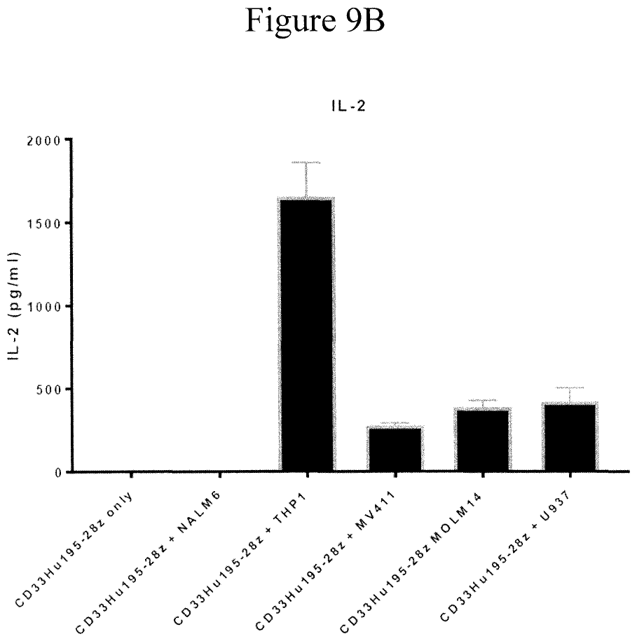

In vitro testing of the constructs revealed that anti-CD33 CD28 CARs consistently produced more IL-2 and Interferon-gamma than anti-CD33 4-1BB CARs across multiple AML cell lines. FIGS. 9A and 9B present results for CD33Hu195-CD28Z CAR.

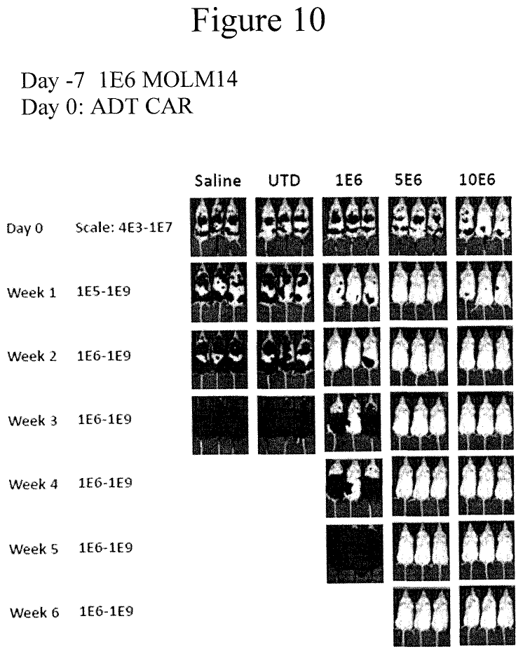

To translate these findings in vivo, xenograft models (mice: NOD.Cg-Prkdcscid Il2rgtm1Wjl/SzJ stock #005557) were injected with MOLM14 AML cells and treated with either anti-CD33 CD28 CAR or anti-CD33 4-1BB CART cells. By bioluminescence imaging, anti-CD33 CD28 CAR-treated mice had no detectable disease while anti-CD33 4-1BB CAR-treated mice presented with leukemia. See FIG. 10.

Combined, the in vitro and in vivo results suggest that the co-stimulatory domain does play a role in CAR T cell functionality and may improve CAR potency.

To confirm the presence of AML in mice detected by bioluminescence, flow cytometry was performed on tissues from mock and anti-CD33 4-1BB CAR-treated mice. No leukemia was found in the bone marrow of mock T cell-treated mice. In contrast, anti-CD33 4-1BB CAR-treated animals were clear of any leukemia in the bone marrow, suggesting the presence of extra medullary disease (EMD). The development of EMD in the less potent anti-CD33 4-1BB CAR-treated mice suggests that CAR immune pressure may be potent enough to clear primary sites of leukemia such as the bone marrow, but unable to eliminate disease in secondary tissues that AML can seed. Treatment of AML with chemotherapy often leads to the development of extramedullary disease in the form of chloromas.

Another AML model, THP1, regularly presents with EMD even in the absence of CAR pressure. With anti-CD33 CD28 CAR against THP1, there was clearance in compartments bone marrow, however anti-CD33 CD28 CAR was not able to prevent the development of EMD. These experiments suggest that although the CD28 costimulatory domain is more potent than 4-1BB in MOLM14, the potency of CD28 is still not able to overcome EMD in all models.

### Example 4

This example demonstrates the use of CARs in accordance with embodiments of the invention.

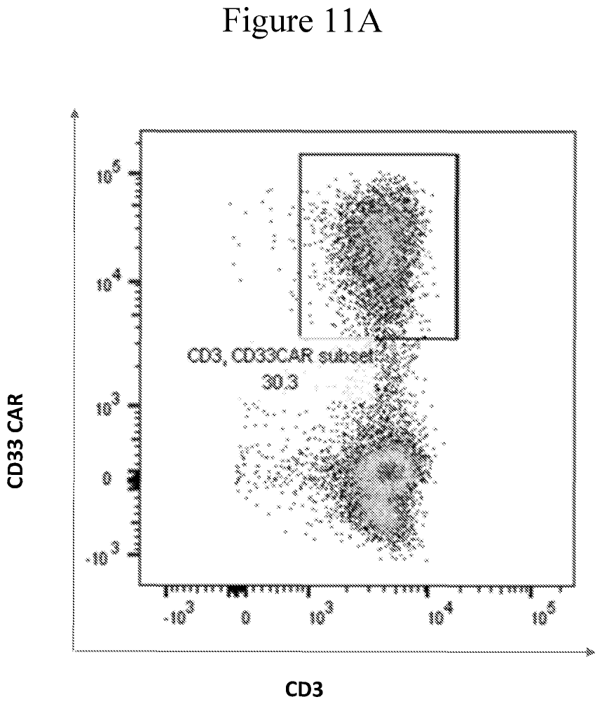

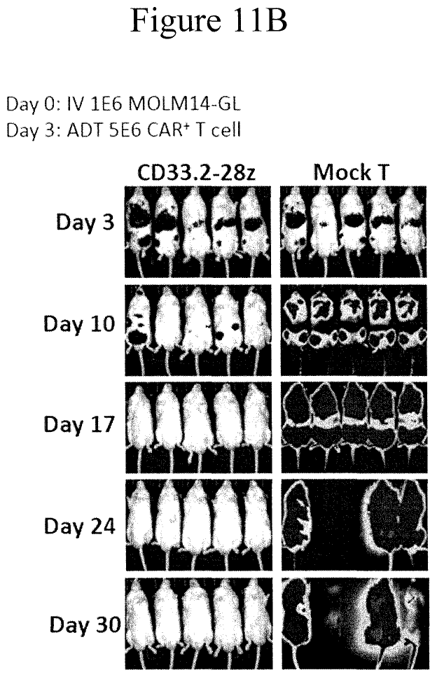

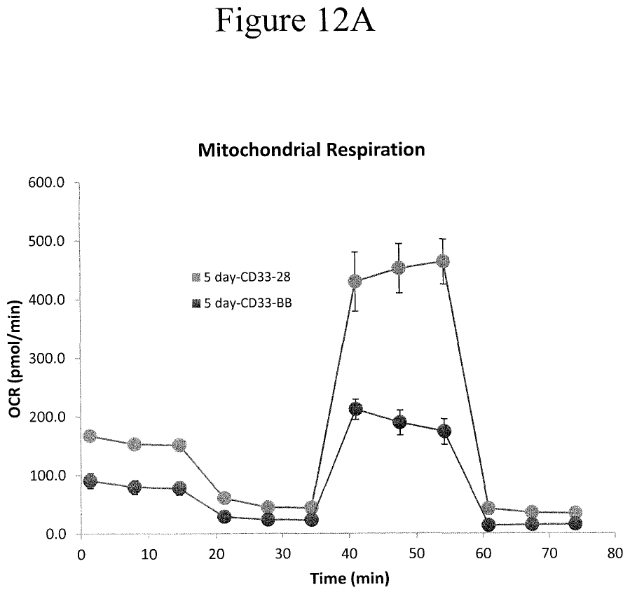

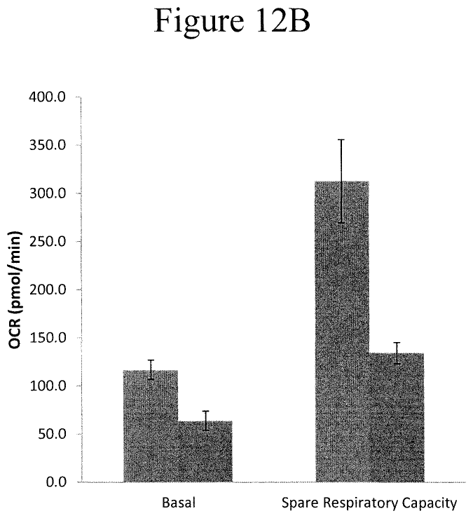

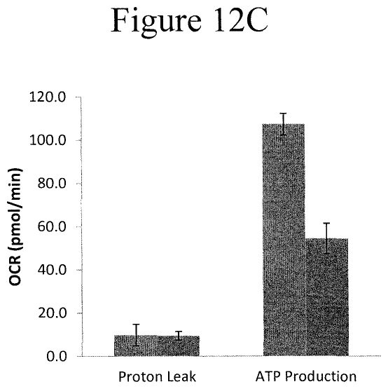

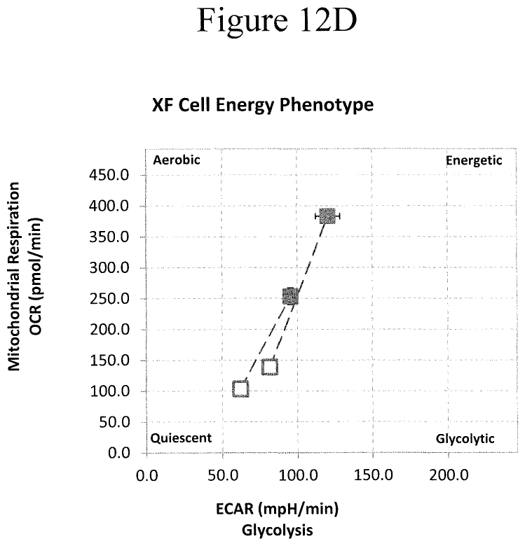

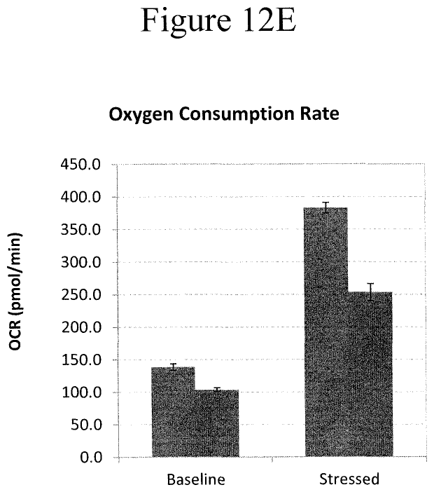

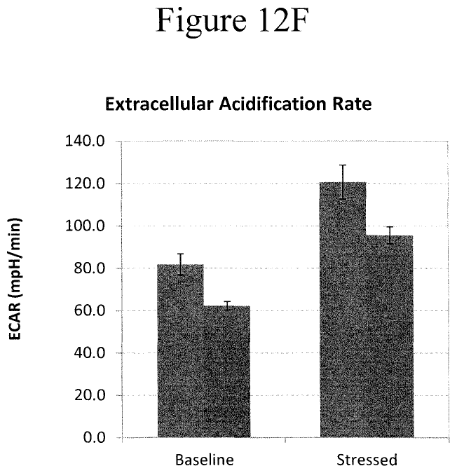

FIGS. 11-12 present additional data.

FIG. 11A: Binding to the biotinalated Siglec-3 confirmed the function of the CD33.2 CAR in vitro.

FIG. 11B: Confirming of the potent activity of CD33.2-28Z in different lenti virus production setting.

FIG. 12A-C: CD33-C28z demonstrated stronger mitochondrial respiration with more spare respiration capacity and better ATP production-linked oxygen consumption rate.

FIG. 12D-F: Surprisingly, CD33-28z also has enhanced glycolytic metabolism with higher extracellular acidification rate.

For CD33 CAR detection with Biotinylated Human Siglec-3/CD33 Protein: CAR detection with: add 2 ul of Biotinylated Human Siglec-3/CD33 Protein, [Avi Tag (Avitag™) Acro Biosystems, Newark, Del., USA], incubate for 20 min, wash once and incubate with 0.5 μl of Streptavidin-PE, incubate for additional 10 min, wash once, subject for FACS analysis.

Principal component analysis of the RNAseq data demonstrated different gene expression profiles associate with CD33.2-28z and CD33.2-BBz CARs at 6 hour or 24 hours post co-incubation with equal number of MOLM14 target cells.

CD33.2-28z demonstrated none or minimal on site off tumor toxicity. 1E5 of CAR′ T cell were co-incubated with equal number of varies of iPS cell lines representing normal tissues. The IFNg level in the culture supernatant were detected with the IFNg kit from R&D.

### Example 5

This example demonstrates the use of CARs in accordance with embodiments of the invention.

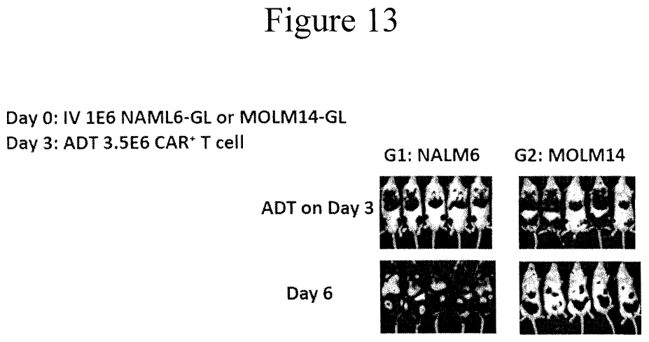

NALM6 was used as a non-CD33 expression tumor model and compared with the MOLM14 model. Treatment was performed using the CD33.2-28z CAR. In the NALM6 model, the tumor continued progression, while in the MOLM14 model, there was reduced reduced tumor burden 3 days later post CAR treatment. See FIG. 13.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A chimeric antigen receptor (CAR) comprising an antigen binding domain having antigenic specificity for CD33, a transmembrane domain, and an intracellular T cell signaling domain, wherein
the antigen binding domain comprises from N-terminus to C-terminus the amino acid sequences of (a) SEQ ID NOS: 15, 4, and 16 or (b) SEQ ID NOS: 13, 4, and 14.

2.-8. (canceled)

9. A chimeric antigen receptor (CAR) comprising an antigen binding domain having antigenic specificity for CD33, a transmembrane domain, and an intracellular T cell signaling domain, wherein
(a) the antigen binding domain comprises the light chain variable CDR1, CDR2, and CDR3 regions of hP67.6; and/or
(b) the antigen binding domain comprises the heavy chain variable CDR1, CDR2, and CDR3 regions of hP67.6
wherein the CDR regions are the amino acid sequences of SEQ ID NOS: 47-52.

10. The CAR according to claim 9, wherein the antigen binding domain comprises the amino acid sequence of SEQ ID NO: 3.

11. The CAR according to claim 9, wherein the antigen binding domain comprises the amino acid sequence of SEQ ID NO: 5.

12. The CAR according to claim 9, wherein the antigen binding domain comprises the amino acid sequence of SEQ ID NO: 4.

13. The CAR according to claim 9, wherein the antigen binding domain comprises the amino acid sequences of SEQ ID NOS: 3, 4, and 5.

14. The CAR according to claim 1, wherein the CAR comprises (i) the amino acid sequence of SEQ ID NO: 7 and the amino acid sequence of SEQ ID NO: 6, or (ii) the amino acid sequence of SEQ ID NO: 11 and the amino acid sequence of SEQ ID NO: 10.

15. The CAR according to claim 1, wherein the CAR comprises the amino acid sequence of SEQ ID NO: 8, the amino acid sequence of SEQ ID NO: 9, or both.

16. The CAR according to claim 1, wherein the CAR further comprises a spacer.

17. A chimeric antigen receptor (CAR) comprising (a) the amino acid sequence of any one of SEQ ID NOS: 18-23 or (b) the amino acid sequence of SEQ ID NO: 23 wherein the amino acid Q within the amino acid sequence of SEQ ID NO: 37 within the amino acid sequence of SEQ ID NO: 23 is substituted with K.

18. A nucleic acid comprising a nucleotide sequence encoding the CAR according to claim 1.

19. The nucleic acid according to claim 18, wherein the nucleotide sequence is codon-optimized.

20. A recombinant expression vector comprising the nucleic acid according to claim 18.

21. An isolated host cell comprising the recombinant expression vector of claim 20.

22. A population of cells comprising at least one host cell of claim 21.

23. A pharmaceutical composition comprising the CAR according to claim 1, and a pharmaceutically acceptable carrier.

24. A method of detecting the presence of cancer, comprising:
(a) contacting a sample comprising one or more cells with the CAR according to claim 1, thereby forming a complex, and
(b) detecting the complex, wherein detection of the complex is indicative of the presence of cancer.

25. The method of claim 24, wherein the cancer is acute myeloid leukemia.

26. A method of treating or preventing cancer in a mammal comprising administering to the mammal the CAR according to claim 1, in the treatment or prevention of cancer in a mammal in an amount effective to treat or prevent cancer in the mammal.

27. The method according to claim 26, wherein the cancer is acute myeloid leukemia.
